(19) United States
(12) Patent Application Publication
(10) Pub. No.: US 2009/0220432 A1
(43) Pub. Date: Sep. 3, 2009

(54) Imaging agents and methods of using same for detecting multidrug resistance in cancer

(75) Inventors: Dimitri Artemov, Timonium, MD (US); Yoshinori Kato, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/991,100

(22) PCT Filed: Aug. 31, 2006
(86) PCT No.: PCT/US06/34461

§ 371 Date: May 4, 2009

Related U.S. Application Data
(60) Provisional application No. 60/712,989, filed on Aug. 31, 2005.

(51) Int. Cl. A61K 49/14; C07K 7/02; A61K 38/15; C12Q 1/02

(52) U.S. Cl. 424/9.34; 530/323; 514/6; 435/29

(57) ABSTRACT

Compounds and methods of using such compounds in the imaging and detection of multidrug resistance cancer in a subject are provided. In particular, novel imaging agents are provided which are suitable for the detection and imaging of a multidrug resistance phenotype in cancer cells and/or tissues using non-invasive medical imaging modalities.

### INCORPORATION BY REFERENCE

This application claims the benefit of U.S. Provisional Application Ser. No. 60/712,989, filed Aug. 31, 2005, the teachings of which are incorporated entirely herein by reference.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### STATEMENT OF RIGHTS TO INVENTIONS MADE UNDER FEDERALLY SPONSORED RESEARCH

This invention was funded, at least in part, by NIH grant R01 CA097310. Accordingly, the U.S. Government may have certain rights to this invention.

### BACKGROUND OF THE INVENTION

1. Field of the Invention

The present invention relates to compounds and methods for using same for the detection of multidrug resistance phenotype in cancer in vivo and in vitro. The present invention in particular relates to diagnosing, detecting and/or monitoring multidrug resistance in a subject having cancer during chemotherapeutic treatment utilizing noninvasive imaging modalities, including magnetic resonance imaging (MRI, nuclear imaging (e.g. SPECT and PET), and optical imaging.

2. Background

Multi-drug resistance (MDR) is a common phenomenon observed in cancer patients who undergo treatment with chemotherapeutic agents. MDR is an acquired drug resistance, in the sense that, after the initial treatment response, the tumor becomes resistant not only to the drug used in the treatment but also to a broad range of structurally and functionally unrelated drugs (Neyfakh, 2002; Bodo et al., 2003). Clinically, the emergence of MDR presents a problem of critical importance, as it significantly reduces the ability to control tumors that become refractive to a wide variety of therapeutic agents.

The molecular mechanisms of MDR include overexpression of ATP binding cassette (ABC) transporters, such as P-glycoprotein (P-gp) MDR1, multi-drug resistance associated proteins (MRPs), and the ABC half-transporter, ABCG2, all of which are localized in the cell plasma membrane (Bodo et al., 2003). MDR1 and ABCG2 pump out large hydrophobic, positively charged molecules while MRPs export both hydrophobic uncharged molecules and water-soluble anionic compounds (Bodo et al., 2003).

Detection of MDR in solid tumors requires subjecting a patient to multiple biopsies, which often is not an acceptable option. In addition, even with modern molecular biology assays, it is not a trivial task to determine the functional status of the tumor drug efflux machinery that is believed to be a major mechanism of MDR.

Alternative and improved methods for detecting MDR in cancer, in particular, during chemotherapeutic therapy, which are less-invasive, more sensitive, and easier to implement are needed in the art. Compounds and methods toward this end would represent an advance in the art.

Citation or identification of any document in this application is not an admission that such document is available as prior art to the present invention.

### SUMMARY OF THE INVENTION

In view of the rapid rise of multidrug resistance in cancer and the substantial obstacles created in the treatment of cancers which develop multidrug resistance, there is an urgent need for new and useful approaches allowing for improved detection, assessment and monitoring of multidrug resistance in subjects before, during or after chemotherapeutic treatments. It would be especially advantageous that such approaches be non-invasive where the multidrug resistance phenotype is being assessed, detected or monitored in vivo. The methods and compounds of the present invention provide a solution, inter alia, to these urgent needs.

The invention disclosed herein provides compounds and methods of using such compounds in the imaging and detection of multidrug resistance cancer in a subject. In particular, the present invention provides novel imaging agents which are suitable for the detection and imaging of a multidrug resistance phenotype in cancer cells and/or tissues using non-invasive medical imaging modalities. The methods and compounds of the invention can advantageously enable a practitioner to modify, adjust and optimize particular chemotherapeutic treatments in response to the presence or absence or the development of multidrug resistance in a subject undergoing, having undergone, or about to undergo, an anticancer therapy. Such opportunities for the modification, adjustment and/or optimization of a treatment provided by the present invention advantageously can prevent or reduce multidrug resistance in a subject undergoing, having undergone, or about to undergo a chemotherapeutic treatment.

The compounds of the invention provide novel imaging agents that can be used to probe and image multidrug resistant cells in vivo or in vitro which advantageously comprise a combination of functional elements allowing for the compound to be internalized into a cell, detected or imaged using a medical imaging modality, and a specifically effluxed from drug resistant cells.

Accordingly, the invention provides in one aspect an agent for imaging a multidrug resistance phenotype in a cancer cell, the agent comprising a transduction domain capable of translocating the agent into the cancer cell, a label domain capable of being detected, and a substrate domain capable of functioning as a substrate for a multidrug resistance transporter such that the compound undergoes specific efflux in a drug resistant cell.

In another aspect, the present invention provides a compound or pharmaceutically acceptable salt thereof according to Formula 1:

[Y¹—Y³]—Y²

wherein Y¹ is a transduction peptide or a “transduction domain,” Y² is a contrast agent or “label domain” capable of being detected by an imaging modality, and Y³ is a multidrag resistance transporter substrate or a “substrate domain.” The three domains can be arranged together as a single conjugate compound by any suitable molecular configuration. The bonds joining the different domains can be covalent or noncovalent. In addition, the different domains can be joined directly to each other or to each other via one or more linker moieties.

The present invention, in another aspect, provides a pharmaceutical composition comprising the compounds of the invention.

In yet another aspect, the present invention provides a method for detecting a cancer cell having a multidrug resistance phenotype comprising, providing an imaging agent, contacting the cancer cell with the imaging agent, and making an image using a medical imaging modality, wherein the imaging agent comprises a transduction domain capable of translocating the agent into the cancer cell, a label domain capable of being detected by the medical imaging modality, and a substrate domain capable of being transported by a multidrug resistance transporter.

In still another aspect, the present invention provides a method for preventing or reducing multidrug resistance cancer in a subject undergoing treatment with a chemotherapeutic agent comprising administering to the subject an imaging agent in an amount sufficient to detect in the subject a multidrug resistance phenotype if present, making an image using a medical imaging modality, reading the image to detect a multidrug resistance phenotype if present, and optimizing the chemotherapeutic treatment if a multidrug resistance phenotype is detected thereby preventing or reducing the multidrug resistant cancer in the subject, wherein the imaging agent comprises a transduction domain capable of translocating the agent into the cancer cell, a label domain capable of being detected by the medical imaging modality, and a substrate domain capable of being transported by a multidrug resistance transporter.

In a further aspect, the step of optimizing the chemotherapeutic treatment comprises administering an inhibitor of the multidrug resistance phenotype, adjusting the treatment with a chemotherapeutic agent such that it becomes effective against the multidrug resistance phenotype, or administering a different chemotherapeutic agent known to be effective against the multidrug resistance phenotype.

In another aspect, the invention provides a method for predicting effectiveness of treatment with a chemotherapeutic agent comprising administering to the subject an imaging agent in an amount sufficient to detect in the subject a multidrug resistance phenotype if present, making an image using a medical imaging modality, reading the image to detect a multidrug resistance phenotype if present, wherein a lack of detected multidrug resistance phenotype predicts an effective treatment with the chemotherapeutic agent, and wherein the imaging agent comprises a transduction domain capable of translocating the agent into the cancer cell, a label domain capable of being detected by the medical imaging modality, and a substrate domain capable of being transported by a multidrug resistance transporter.

In still another aspect, the present invention provides an imaging method comprising the steps of providing an imaging compound according to Formula 1:

[Y¹—Y³]—Y²

wherein, Y¹ is a transduction domain; Y² is a contrast agent capable of being detected by an imaging modality; and Y³ is a multidrug resistance transporter substrate; contacting cells or tissues with the compound; and making an image with an imaging modality.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a depiction of the (A) chemical structure and (B) concept of the Tat-GdDOTA-TAMRA contrast agent. The symbols are as follows, R: Arginine, K: Lysine, Q: Glutamine, G: Glycine. The HIV-1 Tat basic domain peptide is indicated as “(a)”, GdDOTA is indicated as “(b)”, and carboxytetramethylrhodamine (TAMRA) is indicated as “(c).”

FIG. 2 is a scatter plot of rhodamine 123 uptake and P-glycoprotein (P-gp) expression in MCF-7^{wt} and MCF-7^{adr} breast cancer cell lines.

FIG. 3 shows photomicrographs of (A) MCF-7^{wt} and (B) MCF 7^{adr} and (C) cells after treatment with Tat-GdDOTA-TAMRA (0.5 mg/ml) for 20 min. Cells were treated with 100 μg/ml DMCD prior to the exposure to Tat-GdDOTA-TAMRA. An Eclipse E400 microscope with a C-SHG1 super high pressure mercury lamp and a Coolpix 5000 digital camera (Nikon Inc., Tokyo, Japan) was used for fluorescent microscopy. Exposure time= 1/15 s, magnification=×40. The top row shows visible fields and the bottom row shows fluorescent fields.

FIG. 4 is a graph showing fluorescence detection of Tat-GdDOTA-TAMRA taken up by MCF-7^{wt} and MCF-7^{adr} cells after exposure to 0.5 mg/ml of same for 20 min. Dotted lines represent controls (line “a”: MCF-7^{wt}; line “b”: MCF-7^{adr}). Solid lines represent the cells treated with Tat-GdDOTA-TAMRA (line “c”: MCF-7^{wt}; line “d”: MCF-7^{adr}). Line “e” shows the cells treated with 100 μg/ml DMCD prior to exposure to the contrast agent. A FACSCalibur (Becton Dickinson Biosciences, San Jose, Calif., U.S.A.) was used for flow cytometry. Acquisition and analysis were performed with Cell Quest software (Becton Dickinson Biosciences, San Jose, Calif., U.S.A.).

FIG. 5 shows confocal microscopy images of (A) MCF-7^{wt} and (B) MCF-7^{adr} cells treated with Tat-GdDOTA-TAMRA (bright grey fluorescence) as a contrast agent and DAPI (dark grey fluorescence) to visualize nuclei staining.

FIG. 6 shows magnetic resonance imaging of MCF-7^{wt} and MCF-7^{adr} cells treated with the multidrug resistance-specific contrast agent, Tat-GdDOTA-TAMRA. (A) T₁ map. 1: water, 2: MCF-7^{wt} control, 3: MCF-7^{wt} treated with Tat-GdDOTA-TAMRA, 4: MCF-7^{wt} treated with Tat-GdDOTA-TAMRA and cyclosporin A, 5: 0.05 M of GdDTPA, 6: MCF-7^{adr} control, 7: MCF-7^{wt} treated with Tat-GdDOTA-TAMRA, 8: MCF-7^{wt} treated with Tat-GdDOTA-TAMRA and cyclosporin A. (B) Intensity map. Details of the procedure are described in the text.

### DETAILED DESCRIPTION

The present invention relates to novel compounds and methods of using such compounds in the imaging and detection of multiple drug resistance in cancer in a subject. In particular, the present invention provides novel imaging agents which are suitable for detection by an imaging modality for use in detecting and imaging a multiple drug resistance phenotype in cancer cells and/or tissues. The imaging agents of the invention are advantageously designed to comprise a transduction domain, which is capable of translocating the imaging agent into a cancer cell, a label domain, which is capable of being detected by an imaging modality, and a substrate domain, which is capable of functioning as a substrate for a multidrug resistance transporter. The present invention further provides pharmaceutical compositions comprising the compounds of the invention for use in detecting or evaluating cells and/or tissues for a multidrug resistance phenotype before or during a treatment using one or more chemotherapeutic agents. The present inventors have conceived and discovered for the first time the advantageous combination of the different functional domains of the imaging compounds of the invention and their use in detecting, evaluating, and monitoring multidrug resistance in a subject or in tissues and/cells before, during or after chemotherapeutic treatments. The use of the compounds and methods of the invention, in one aspect, can provide the opportunity to modify, alter or optimize a particular chemotherapeutic treatment through the assessment and detection of multidrug resistance in a subject. The compounds and methods of the invention can also provide the opportunity to predict the effectiveness of any given chemotherapeutic treatment through the detection and monitoring of a subject for the multidrug resistance phenotype.

### DEFINITIONS

Unless defined otherwise, all technical and scientific terms used herein have the meaning commonly understood by one of ordinary skill in the art to which this invention belongs. The following references can provide one of skill in the art to which this invention pertains with a general definition of many of the terms used in this invention, and can be referenced and used so long as such definitions are consistent the meaning commonly understood in the art: Singleton et al., Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2^{nd} Edition, W.B. Saunders Company. Any additional technical resources available to the person of ordinary skill in the art providing definitions of terms used herein having the meaning commonly understood in the art can be consulted. For the purposes of the present invention, the following terms are further defined. Additional terms are defined elsewhere in the description.

As used herein, the expression “multidrug resistance phenotype” or “MDR phenotype” can refer to the phenotype acquired by a cancer cell, either spontaneously or in response to treatment with a chemotherapeutic agent, which renders the cell simultaneously resistant to a multitude of structurally heterogenous cytotoxic compounds. The particular molecular and/or genetic basis for the MDR phenotype is not intended to limit the herewith meaning, i.e. the invention encompasses any cancer-related MDR phenotype resulting from any underlying genetic or molecular mechanism. For example, the MDR phenotype can be associated with overexpression of P-glycoprotein (MDR1 170 kDa gene product ABC Transporter or ATP binding cassette transporter) or overexpression of the multidrug resistance associated protein, MRP, a 190 kDa multispanning transmembrane protein ABC Transporter or ATP binding cassette transporter.

As used herein, an “inhibitor of the multidrug resistance phenotype” refers to a compound or substance, such as, for example, a small molecule inhibitor, protein/peptide inhibitor or antibody, which prohibits, alleviates, ameliorates, halts, restrains, slows or reverses the progression of or the development of a multidrug phenotype.

Similarly, as used herein, the expression “preventing or reducing multidrug resistance” refers to the prohibition, alleviation, amelioration, halting, restraining, slowing or reversing the progression of development of a multidrug phenotype.

As used herein, the expression “an agent for imaging” or “an imaging agent” or the like refers to the multi-domain conjugate compounds of the invention as described herein, comprising a transduction domain, a label domain and a MDR substrate domain and which can be used to detect, image and/or diagnose a cancer cell having a multidrug resistance phenotype. The imaging agents of the invention advantageously couple together different functional groups capable of directing cell-permeation of the imaging agent (transduction domain) into a cell, transporting the agent out of cells having a multidrug resistant phenotype vis-a-vis an MDR associated efflux pump (or MDR transporter) (the substrate domain), and imaging the compound by way of a noninvasive medical imaging modality, including MRI or nuclear imaging (label domain).

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as sodium, calcium, magnesium, or potassium hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

As used herein, the expression “an amount sufficient to detect a multidrug resistance phenotype” or similar expressions refer to at least the minimum amount of imaging agent of the invention that must be administered to be able to detect or image a cancer cell or tissue having a multidrug resistance phenotype.

As used herein, the expression “making an image” refers to the use of a medical imaging modality, including magnetic resonance imaging, nuclear imaging (i.e., imaging using radiolabeled compounds), optical imaging or the like, to obtain an image of a cell or tissue treated with or having been administered an imaging agent of the invention. Making an image can include the use of a computer system and/or software. The image can be in any readable form, such as, a digital format. Obtaining, manipulating and reading such images are completely within the capacity of the skilled artisan.

As used herein, the term “medical imaging modality” or “imaging modality” refers to the variety of different types of medical imaging systems for imaging biological cells and tissues, e.g. magnetic resonance imaging, nuclear imaging (PET of SPECT), ultrasound, x-ray or the like. A broad range of capabilities and features are typically offered in each imaging modality. For example, a magnetic resonance imaging (“MRI”) system may be offered with a range of polarizing magnetic strengths and configurations and with a range of different optional features such as magnetic resonance angiography (“MRA”), cardiac imaging and functional magnetic resonance imaging (“fMRI”). Despite the many differences, medical imaging systems have a number of basic functions in common. All medical imaging systems include an operator interface which enables a particular image acquisition to be prescribed, a data acquisition apparatus which uses one of the imaging modalities to acquire data from the subject, an image reconstruction processor for reconstructing an image using acquired data, and storage apparatus for storing images and associated patient information. Typically, hardware is designed to carry out these functions and software is designed and written for each hardware configuration.

It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### Multidrug Resistance

The person of ordinary skill in the art will appreciate that the following general description of multidrug resistant cancer is not meant to limit the herewith disclosed invention nor should it be construed as an admission of prior art.

Many human cancers intrinsically express or spontaneously develop resistance to several classes of anticancer drugs at the same time, notwithstanding that each of the drug classes have different structures and mechanisms of action. This phenomenon, which can be mimicked in cultured mammalian cells, is generally referred to as multidrug resistance (“MDR”) or the multidrug resistance phenotype. The MDR phenotype presents significant obstacles to the successful chemotherapeutic treatments for cancers in human patients. Resistance of malignant tumors to multiple chemotherapeutic agents is a major cause of treatment failure (Wittes et al., Cancer Treat. Rep. 70:105 (1986); Bradley, G. et al., Biochim. Biophys. Acta 948:87 (1988); Griswald, D. P. et al., Cancer Treat. Rep. 65(S2):51 (1981); Osteen, R. T. (ed.), Cancer Manual, (1990)). Tumors initially sensitive to cytotoxic agents often recur or become refractory to multiple chemotherapeutic drugs (Riordan et al., Pharmacol. Ther. 28:51 (1985); Gottesman et al., Trends Pharmacol. Sci. 9:54 (1988); Moscow et al., J. Natl. Cancer Inst. 80:14 (1988); Croop, J. M. et al., J. Clin. Invest. 81:1303 (1988)). Cells or tissues obtained from tumors and grown in the presence of a selecting cytotoxic drug can result in cross-resistance to other drugs in that class as well as other classes of drugs including, but not limited to, anthracyclines, Vinca alkaloids, and epipodophyllotoxins (Riordan et al., Pharmacol. Ther. 28:51 (1985); Gottesman et al., J. Biol. Chem. 263:12163 (1988)). Thus, acquired resistance to a single drug results in simultaneous resistance to a diverse group of drugs that are structurally and functionally unrelated. Such resistance can be a problem for both solid-form and liquid-form tumors (e.g. blood or lymph-based cancers).

The characteristics of the multidrug resistance phenotype have been analyzed by studies on normal and tumor cell lines isolated for resistance to selected cytotoxic drugs. One major mechanism of multidrug resistance in mammalian cells involves the increased expression of the 170 kDa plasma membrane glycoprotein pump system (Juranka et al., FASEB J 3:2583 (1989); Bradley, G. et al., Blochem. Biophys. Acta 948:87 (1988)). The gene encoding this pump system, sometimes referred to as a multidrug transporter, has been cloned from cultured human cells and is generally referred to as mdr1 or MDR1. This gene is expressed in several classes of normal tissues, but physiological substrates transported for the mdr1 gene product in these tissues have not been identified. The MDR1 product is a member of the ABC Transporter Protein superfamily, a group of proteins having energy-dependent export function.

The protein product of the mdr1 gene, generally known as P-glycoprotein (“P-170”, “P-gp”), is a 170 kDa trans-plasma membrane protein that constitutes the aforementioned energy-dependent efflux pump. Expression of P-gp on the cell surface is sufficient to render cells resistant to multiple cytotoxic drugs, including many anti-cancer agents. P-gp-mediated MDR appears to be an important clinical component of tumor resistance in tumors of different types, and mdr1 gene expression correlates with resistance to chemotherapy in different types of cancer.

The nucleotide sequence of the mdr1 gene (Gros, P. et al., Cell 47:371 (1986); Chen, C. et al., Cell 47:381 (1986)) indicates that it encodes a polypeptide similar or identical to P-glycoprotein and that these are members of the highly conserved class of membrane proteins similar to bacterial transporters and involved in normal physiological transport processes. Sequence analysis of the mdr1 gene indicates that Pgp consists of 1280 amino acids distributed between two homologous (43% identity) halves. Each half of the molecule has six hydrophobic transmembrane domains and each has an ATP binding site within the large cytoplasmic loops. Only about 8% of the molecule is extracellular, and the carbohydrate moiety (approximately 30 kDa) is bound to sites in this region.

Thus, it will be appreciated that mammalian cells having a “multidrug-resistance” or “multidrug-resistant” phenotype are characterized by the ability to sequester, export or expel a plurality of cytotoxic substances (e.g., chemotherapeutic drugs) from the intracellular milieu. Cells may acquire this phenotype as a result of selection pressure imposed by exposure to a single chemotherapeutic drug (the selection toxin). Alternatively, cells may exhibit the phenotype prior to toxin exposure, since the export of cytotoxic substances may involve a mechanism in common with normal export of cellular secretion products, metabolites, and the like. Multidrug resistance differs from simple acquired resistance to the selection toxin in that the cell acquires competence to export additional cytotoxins (other chemotherapeutic drugs) to which the cell was not previously exposed. For example, Mirski et al. (1987), 47 Cancer Res. 2594-2598, describe the isolation of a multidrug-resistant cell population by culturing the H69 cell line, derived from a human small cell lung carcinoma, in the presence of adriamycin (doxorubicin) as a selection toxin. Surviving cells were found to resist the cytotoxic effects of anthracycline analogs (e.g., daunomycin, epirubicin, menogaril and mitoxantrone), acivicin, etoposide, gramicidin D, colchicine and Vinca-derived alkaloids (vincristine and vinblastine) as well as of adriamycin. Similar selection culturing techniques can be applied to generate additional multidrug-resistant cell populations.

As mentioned above, the functional property of multidrug-resistance is associated with expression and cell-surface display of one or more ABC Transporter Protein superfamily members (e.g. ATP binding cassette transporters) with energy-dependent export function (e.g., P-glycoprotein, MRP). The cell population described in Mirski et al. (1987) was reported in Cole et al. (1992), 258 Science 1650-1654 to overexpress MRP (a correction of the reported MRP sequence appears at 260 Science 879). Currently, antibodies specifically reactive with P-glycoprotein or MRP, or nucleic acid probes specific for the corresponding expressed nucleic acid sequences, are used to ascertain the molecular basis of multidrug-resistance in a given cell population. Where the cell population in question includes transformed cells in the body of a cancer sufferer, determination of the molecular basis of the observed phenotype can assist the clinician in ascertaining whether treatment with one of the so-called “chemosensitizers” or “MDR reversal agents,” the majority of which affect P-glycoprotein, is appropriate. Thus, knowledge of the molecular basis of the observed phenotype provides information relevant to developing or revising a course of disease management. Zaman et al. (1993), 53 Cancer Res. 1747-1750, cautions, however, that the induction or overexpression of MRP does not account for all forms of multidrug-resistance phenotype that are not attributable to P-glycoprotein expression. It will be appreciated that additional members of the ABC Transporter Protein family may exist in the mammalian (e.g., human) genome and likely contribute to the occurrence of multidrug-resistance in transformed cells. The methods and compounds of the present invention are applicable to any form of multidrug resistance, whether or not the exact molecular mechanism is known or fully understood.

In view of the above, the skilled artisan will appreciate that the phenomenon of multidrug resistance in cancer is an important and significant problem facing the medical field today and which impacts the lives of millions of cancer patients. Researchers throughout the world continue to press for techniques for understanding the multidrug resistance problem and for improving anticancer treatments susceptible thereto. The present inventors have discovered new and useful imaging agents and methods of using same which can advantageously be used with non-invasive imaging modalities, especially MRI, to detect, monitor and evaluate the state of multidrug resistance in a subject suffering from cancer, especially during an anticancer treatment such that the treatment can be modified, altered and/or improved depending on the status of the multidrug resistance.

### Imaging Agents

3In one aspect, the present invention provides novel compounds for detecting and/or d333iagnosing and/or imaging an MDR resistance phenotype in a cancer cell and/or tissue which are compatible with noninvasive imaging modalities, including, but not limited to MRI or nuclear imaging (e.g. SPECT or PET). In a further aspect, the compounds of the invention can comprise a transduction domain, which is capable of translocating the compound into a cancer cell, a label domain, which is capable of being detected by a noninvasive imaging modality, and a substrate domain, which is capable of functioning as a substrate for a transporter associated with a multidrug resistance phenotype (herein sometimes as an “MDR substrate domain”). It is the discovery of the present inventors that the novel combination of these three domains, namely, a transduction domain, a label domain, and an MDR substrate domain, can unexpectedly provide a useful and effective means for detecting and/or imaging and/or diagnosing a multidrug resistance phenotype, in vivo or in vitro, using noninvasive medical imaging modalities, which can include, for example, MRI, nuclear imaging (PET or SPECT), optical imaging, sonoluminence or photoacoustic imaging.

In another aspect, the inventive compounds of the present invention can be represented by the structure of Formula 1:

[Y¹—Y³]—Y²

wherein Y¹ is a transduction peptide or a “transduction domain,” Y² is a contrast agent or “label domain” capable of being detected by an imaging modality, and Y³ is a multidrug resistance transporter substrate or a “substrate domain.” The three domains can be arranged together as a single conjugate compound by any suitable molecular configuration. The bonds joining the different domains can be covalent or noncovalent. In addition, the different domains can be joined directly to each other or to each other via one or more linker moieties.

The term “transduction domain,” as used herein, refers to any compound that is capable of translocating the imaging agent across a cellular membrane (or organellar membrane) of a cancer cell. Transduction domains are known in the art and can include, for example, an amphiphilic membrane translocation peptide, such as, for example, HIV-1 Tat basic domain peptide. By “protein transduction domain” or “transduction domain”, it is meant an amino acid sequence that facilitates protein entry into a cell or cell organelle. Exemplary protein transduction domains include but are not limited to a minimal unidecapeptide protein transduction domain (corresponding to residues 47-57 of HIV-1 TAT comprising YGRKKRRQRRR), a polyarginine sequence comprising a number of arginines sufficient to direct entry into a cell (e.g., 3, 4, 5, 6, 7, 8 or 9 arginines), a VP22 domain (Zender et al., Cancer Gene Ther. 2002 June; 9(6):489-96), an Drosophila Antennapedia protein transduction domain (Noguchi et al., Diabetes 2003; 52(7):1732-1737), a truncated human calcitonin peptide (Trehin et al. Pharm. Research, 21:1248-1256, 2004), polyarginine (e.g. a 16-mer) (Allen et al. J. Biol. Inorg. Chem., 8:246-750, 2003) and polylysine (Wender et al., PNAS, Vol. 97:13003-13008). See, also, Nat Biotechnol. 2001 December; 19(12):1173-6.

Protein transduction domains (also known in the art as “cell-penetrating peptides” or CPPs) (see Richard et al. J. Biol. Chem., 278:585-590, 2003) are short peptide sequences that enable proteins to translocate across the cell and nuclear membranes, leading to entry into the cytosol by means of atypical secretory and internalization pathways (Joliot et al., Nat Cell Biol 2004; 6(3):189-196). In 1988 Green and Loewenstein discovered that the human immunodeficiency virus type 1 (HIV-1) TAT-protein, an 86-amino acid protein, could rapidly enter cells and was even capable of entering the cell nucleus (Green and Loewenstein P M. Cell 1988; 55(6):1179-1188). Building on this observation, a minimal unidecapeptide protein transduction domain (corresponding to residues 47-57 of HIV-1 TAT) was developed by Dowdy and co-workers (Schwarze et al., Science 1999; 285(5433):1569-1572). This unidecapeptide sequence was used successfully to deliver an NH2-terminal TAT-β-galactosidase fusion protein (120 kDa) to mouse tissues via intraperitoneal injections into mice (Schwarze et al., Science 1999; 285(5433):1569-1572). The TAT-β-galactosidase fusion protein retained biological activity. This general method has now been successfully used for the transduction of a variety of proteins, peptides, peptide nucleic acids, antisense oligonucleotides, nanoparticles and liposomes (see Richard et al. J. Biol. Chem., 278:585-590, 2003). PTD-containing peptides or proteins are taken up by cells within 5 minutes at concentrations as low as 100 nM as assessed by direct labeling with fluorescein or by indirect immunofluorescence using antibodies. This uptake is independent of endocytotic mechanisms, transmembrane protein channels, and protein receptor binding. In addition, in vitro studies have demonstrated that protein transduction domain-mediated translocation occurs at low temperatures and exhibits no strong cellular specificity, i.e. CPPs can be internalized in most cell types. The mechanism of internalization of CPPs and/or their cargo is not well understood and somewhat controversial (Richards et al., 2003). Each of the above references relating to transduction domains is herein expressly incorporated in their entirety by reference.

The term “label domain” as used herein refers to a moiety capable of being detected by a noninvasive medical imaging modality, such as, for example, magnetic resonance imaging (MRI), nuclear imaging using radiolabels (e.g., PET or SPECT), or optical imaging or any similarly suitable technique. While certain aspects of the invention relate to the detection and/or imaging of multidrug resistance phenotypes using any imaging technique, including nuclear imaging techniques, in a preferred embodiment, the compounds of the present invention are advantageously compatible with MRI, i.e. utilizing MR contrast agents. Nuclear imaging of MDR using SPECT (a nuclear imaging modality) can also be used with the present invention, however, due to the need for radiopharmaceuticals and the relatively low spatial resolution of SPECT, the lack of anatomical information, and the potentially high doses of radiation from multiple examinations are problems with this technique. Further, while PET imaging can also be used with the present invention, the short half-life of PET tracers significantly narrows the imaging window for experiments, which may not be optimal for detecting the uptake kinetics of MDR-specific agents.

Magnetic resonance imaging (MRI) provides high spatial resolution and excellent anatomical information. It also does not involve exposure to ionizing radiation, thus minimizing the associated risk to the patient. The present invention provides, in part, novel MRI contrast agents for specific imaging of MDR effects in cancer cells. In certain embodiments, the compounds of the invention for MDR detection can be designed and optimized using three functional domains. The first domain, “the transduction domain,” which in one embodiment is the amphiphilic membrane translocation HIV-1 Tat basic domain peptide, facilitates internalization of the agent across the plasma membrane of the cell. The second domain, “the label domain,” which in one embodiment is a GdDOTA chelate complex, generates T₁ MR contrast by reducing the T₁ relaxation time of multiple water molecules interacting with the paramagnetic metal. The third domain, “the substrate domain,” is a substrate for a MDR transporter, and also provides specific efflux of the agent from MDR resistant cancer cells. In one embodiment, the substrate domain is the rhodamine fluorescent dye carboxytetramethylrhodamine (TAMRA), which is a substrate for P-glycoprotein. An additional benefit of using a fluorescent compound as the substrate domain of the compounds of the invention is that the inventive agents can be detected by both MRI and optical fluorescent imaging techniques, such as flow cytometry (FACS) and fluorescent/confocal microscopy.

The different medical imaging modalities of the present invention, their uses and operations, will be well-known to the skilled artisan. It will be particularly appreciated that magnetic resonance imaging (MRI) is a clinically important medical imaging modality due to its exceptional soft-issue contrast. MRI scanners use the technique of nuclear magnetic resonance (NMR) to induce and detect a very weak radiofrequency signal that is a manifestation of nuclear magnetism. The term “nuclear magnetism” refers to weak magnetic properties that are exhibited by some materials as a consequence of the nuclear spin that is associated with their atomic nuclei. In particular, the proton, which is the nucleus of the hydrogen atom, possesses a nonzero nuclear spin and is an excellent source of NMR signals. The human body contains enormous numbers of hydrogen atoms, especially in water and lipid molecules.

In MRI imaging, the patient to be imaged must be placed in an environment in which several different magnetic fields can be simultaneously or sequentially applied to elicit the desired NMR signal. Commercially-available MRI scanners utilize a strong static field magnet in conjunction with a sophisticated set of gradient coils and radiofrequency coils. The gradients and the radiofrequency components are switched on and off in a precisely timed pattern, or pulse sequence. Different pulse sequences are used to extract different types of data from the patient.

After scanning, MRI systems provide a variety of mechanisms to create image contrast. If magnetic resonance images were otherwise restricted to water density, MRI would be considerably less useful, since most tissues would appear identical. Fortunately, many different MRI contrast mechanisms can be employed to distinguish between different tissues and disease processes.

The primary contrast mechanisms exploit the magnetization relaxation phenomena. The two types of relaxations are termed spin-lattice relaxation, characterized by a relaxation time T₁, and spin-spin relaxation, characterized by a relaxation time T₂.

Spin-lattice relaxation describes the rate of recovery of magnetization toward equilibrium after it has been disturbed by radiofrequency pulses. White matter has a shorter T₁ than gray matter, so it produces a stronger signal. The stronger signals then shows up brighter in an image. Because the image highlights the parts with shorter T₁, the image is “T₁-weighted.”

Spin-spin relaxation describes the rate at which the NMR signal decays after it has been created. The signal is proportional to the transverse magnetization. White matter has a shorter T₂ than gray matter, so it produces a weaker signal. Conversely, cerebrospinal fluid (CSF) has a long T₂ and produces more signal. The stronger signals then shows up brighter in an image. Because the image highlights the parts with longer T₂, the image is “T₂-weighted.”

In other embodiments, the methods of the present invention will utilize nuclear imaging techniques to image and/or detect the imaging compounds of the invention. In such embodiments, the label domain of the imaging compounds of the invention can comprise a radionuclide reporter appropriate for scintigraphy, SPECT, or PET imaging or equivalent nuclear imaging technologies. For use with PET, the label domain comprises one of the various positron emitting metal ions, such as ⁵¹Mn, ⁵²Fe, ⁶⁰Cu, ⁶⁸Ga, ⁷²As, ^{94m}Tc, or ¹¹⁰In. Preferred metal radionuclides include ⁹⁰Y, ^{99m}Tc, ¹¹¹In, ⁴⁷Sc, ⁶⁷Ga, ⁵¹Cr, ¹⁷⁷mSn, ⁶⁷Cu, ¹⁶⁷Tm, ⁹⁷Ru, ¹⁸⁸Re, ¹⁷⁷Lu, ¹⁹⁹Au, ²⁰³Pb, and ¹⁴¹Ce. ^{99m}Tc is preferred because of its low cost, availability, imaging properties, and high specific activity. The nuclear and radioactive properties of Tc-99m make this isotope an ideal scintigraphic imaging agent. This isotope has a single photon energy of 140 keV and a radioactive half-life of about 6 hours, and is readily available from a ⁹⁹Mo-^{99m}Tc generator. The radioactive metals may be chelated by, for example, linear, macrocyclic, terpyridine, and N³S, N²S², or N⁴ chelants (see also, U.S. Pat. Nos. 5,367,080, 5,364,613, 5,021,556, 5,075,099, 5,886,142, incorporated herein by reference), and other chelators known in the art including, but not limited to, HYNIC, DTPA, EDTA, DOTA, TETA, DTPA and bisamino bisthiol (BAT) chelators (see also U.S. Pat. No. 5,720,934, incorporated by reference). The radioactive metals can also be chelated by more than one chelator. The chelates may be covalently linked directly to one or both of the other domains of the imaging agents, e.g. the transduction and substrate domains, or linked to one or both of those domains via a linker, as described herein, and then directly labeled with the radioactive metal of choice (see, WO 98/52618, U.S. Pat. Nos. 5,879,658, and 5,849,261, incorporated herein by reference).

In forming the imaging agents and/or the label domain of the invention comprising radioactive technetium, the technetium complex, which can be a salt of Tc-99m pertechnetate, can be reacted with the reagent in the presence of a reducing agent. Preferred reducing agents are dithionite, stannous and ferrous ions; one preferred reducing agent is stannous chloride. Means for preparing such complexes can be conveniently provided in a kit form comprising a sealed vial containing a predetermined quantity of a reagent of the invention to be labeled and a sufficient amount of reducing agent to label the reagent with Tc-99m. Alternatively, the complex may be formed by reacting a peptide of this invention (e.g. a transduction peptide domain) conjugated with an appropriate chelator with a pre-formed labile complex of technetium and another compound known as a transfer ligand. This process is known as ligand exchange and is well known to those skilled in the art. The labile complex may be formed using such transfer ligands as tartrate, citrate, gluconate or mannitol, for example. Among the Tc-99m pertechnetate salts useful with the present invention are included the alkali metal salts such as the sodium salt, or ammonium salts or lower alkyl ammonium salts.

Radioactively-labeled scintigraphic imaging agents provided by the present invention can be provided having a suitable amount of radioactivity. In forming Tc-99m radioactive complexes, it is generally preferred to form radioactive complexes in solutions containing radioactivity at concentrations of from about 0.01 millicurie (mCi) to 100 mCi per mL.

Generally, the unit dose to be administered has a radioactivity of about 0.01 mCi to about 100 mCi, preferably 1 mCi to 20 mCi. The solution to be injected at unit dosage is from about 0.01 mL to about 10 mL.

Typical doses of a radionuclide-labeled imaging agents according to the invention can provide 10-20 mCi. After injection of the radionuclide-labeled imaging agent into a subject, a gamma camera calibrated for the gamma ray energy of the nuclide incorporated in the imaging agent can be used to image areas of uptake of the agent and quantify the amount of radioactivity present at the site of uptake. Imaging in vivo can take place in a matter of a few minutes. However, imaging can take place, if desired, in hours or even longer, after the radiolabeled peptide is injected into a patient. In most instances, a sufficient amount of the administered dose will accumulate in the area to be imaged within about 0.1 of an hour to permit the taking of scintiphotos.

The present invention further contemplates imaging agents that are compatible with addition noninvasive medical imaging technologies, such as, optical imaging, sonoluminescence or photoacoustic imaging. In accordance with the present invention, a number of optical parameters may be employed to detect the MDR phenotypes with in vivo light imaging after injection of the subject with an optically-labeled imaging agent, i.e. where the label domain is a moiety compatible with optical imaging technology. Optical parameters to be detected in the preparation of an image may include transmitted radiation, absorption, fluorescent or phosphorescent emission, light reflection, changes in absorbance amplitude or maxima, and elastically scattered radiation. For example, biological tissue is relatively translucent to light in the near infrared (NIR) wavelength range of 650-1000 nm. NIR radiation can penetrate tissue up to several centimeters, permitting the use of the fibrin binding moieties of the present invention for optical imaging of fibrin in vivo. The imaging agent components, e.g. the transduction and/or substrate domains, may be conjugated with photolabels, such as optical dyes, including organic chromophores or fluorophores, having extensive delocalized ring systems and having absorption or emission maxima in the range of 400-1500 nm. The imaging agents of the invention may alternatively be derivatized with a bioluminescent molecule. The preferred range of absorption maxima for photolabels is between 600 and 1000 nm to minimize interference with the signal from hemoglobin. Preferably, photoabsorption labels have large molar absorptivities, e.g. >10⁵ cm⁻¹M⁻¹, while fluorescent optical dyes will have high quantum yields. Examples of optical dyes include, but are not limited to those described in WO 98/18497, WO 98/18496, WO 98/18495, WO 98/18498, WO 98/53857, WO 96/17628, WO 97/18841, WO 96/23524, WO 98/47538, and references cited therein, each of which are incorporated herein by reference. The photolabels may be covalently linked directly to the imaging agent domains, e.g. the transduction or substrate domains, or linked indirectly thereto via a linker, as described herein.

After injection of the optically-labeled imaging agent, the patient can be scanned with one or more light sources (e.g., a laser) in the wavelength range appropriate for the photolabel employed in the agent. The light used may be monochromatic or polychromatic and continuous or pulsed. Transmitted, scattered, or reflected light is detected via a photodetector tuned to one or multiple wavelengths to determine the location of fibrin in the subject. Changes in the optical parameter may be monitored over time to detect the multidrug resistance phenotype in target cancer cells and/or tissues. Standard image processing and detecting devices may be used in conjunction with the optical imaging reagents of the present invention.

The optical imaging reagents described above may also be used for acousto-optical or sonoluminescent imaging performed with optically-labeled imaging agents (see, U.S. Pat. No. 5,171,298, WO 98/57666, and references therein). In acousto-optical imaging, ultrasound radiation is applied to the subject and affects the optical parameters of the transmitted, emitted, or reflected light. In sonoluminescent imaging, the applied ultrasound actually generates the light detected. Suitable imaging methods using such techniques are described in WO 98/57666, which is incorporated herein by reference.

Also within the ambit of the present invention are imaging agents that are compatible with ultrasound imaging. When ultrasound is transmitted through a substance, the acoustic properties of the substance will depend upon the velocity of the transmissions and the density of the substance. Changes in the acoustic properties will be most prominent at the interface of different substances (solids, liquids, gases). Ultrasound contrast agents are intense sound wave reflectors because of the acoustic differences between liquid (e.g., blood) and gas-containing microbubbles, liposomes, or microspheres dissolved therein. Because of their size, ultrasound microbubbles, liposomes, microspheres, and the like may remain for a longer time in the blood stream after injection than other detectable moieties.

In this aspect of the invention, the imaging agents of the invention may be linked to a material which is useful for ultrasound imaging. The materials are employed to form vesicles (e.g., liposomes, microbubbles, microspheres, or emulsions) containing a liquid or gas which functions as the detectable label (e.g., an echogenic gas or material capable of generating an echogenic gas). Materials for the preparation of such vesicles include surfactants, lipids, sphingolipids, oligolipids, phospholipids, proteins, polypeptides, carbohydrates, and synthetic or natural polymeric materials. See, for further description of suitable materials and methods, WO 98/53857, WO 98/18498, WO 98/18495, WO 98/18497, WO 98/18496, and WO 98/18501.

Suitable gases include, but are not limited to, C₁₋₆ perfluorocarbon gases, SF₆, low molecular weight C₁₋₆ fluorinated or halogenated alkenes, alkynes, or cyclized versions of the same, or other suitable gases or mixtures thereof, as described in WO 97/29783, WO 98/53857, WO 98/18498, WO 98/18495, WO 98/18496, WO 98/18497, WO 98/18501, WO 98/05364, WO 98/17324, each of which are incorporated herein by reference. The ultrasound vesicles may be used as is or stabilized with surfactants or some other stabilizing material such as emulsifying agents and/or viscosity enhancers, cryoprotectants, lyoprotectants, or bulking agents.

Since ultrasound vesicles may be larger than the other detectable labels described herein, and thus may be too large in size to be translocated through the cell membrane and then effluxed out by the MDR translocator, the ultrasound-based imaging agents may be used together with at least one other imaging agent having a label domain of another type, e.g. an MR contrast agent or radionuclide complex. In this way, the ultrasound-based imaging agents can be used as a means to locate noninvasively the site of the drug-resistant cancer. Attachment may be via direct covalent bond between the imaging agent and the material used to make the vesicle or via a linker, as described previously. For example, see WO 98/53857 generally for a description of the attachment of a peptide to a bifunctional PEG linker, which is then reacted with a liposome composition. See also, Lanza et al., Ultrasound in Med. & Bio., 23(6): 863-870 (1997). The targeted ultrasound vesicles may be prepared using conventional methods known in the art. Known methods include gentle shaking, rotor mixing, sonication, high pressure homogenization, high speed stirring, high shear mixing, emulsification, and colloidal mill procedures, in the presence or absence of the desired echogenic gas or gas mixture, to generate the vesicles. The desired echogenic gas may alternatively be incorporated into the vesicles by applying an atmosphere or overpressure of said gas to the vesicles (see U.S. Pat. No. 5,674,469).

Ultrasound imaging techniques which may be used in accordance with the present invention include known techniques, such as color Doppler, power Doppler, Doppler amplitude, stimulated acoustic imaging, and two- or three-dimensional imaging techniques. Imaging may be done in harmonic (resonant frequency) or fundamental modes, with the second harmonic preferred.

In view of the above, the imaging agents of the invention are not limited in their compatibility with known medical imaging modalities. A particular preferred embodiment uses imaging agents that are compatible with MRI, as described herein.

Thus, in one aspect, the invention provides an agent for imaging a multidrug resistance phenotype in a cancer cell, the agent comprising a transduction domain capable of translocating the agent into the cancer cell, a label domain capable of being detected, and a substrate domain capable of functioning as a substrate for a multidrug resistance transporter, wherein the label domain is a magnetic resonance contrast agent suitable for magnetic resonance imaging. The contrast agent can comprise a paramagnetic metal atom, typically as a chelate, such as, for example, a chelated gadolinium atom. The chelate can comprise a metal selected from the group consisting of Gd(III), Fe(III), Mn(II and III), Cr(III), Cu(II), Dy(III), Tb(III), Ho(III), Er(III), and Eu(III). In addition, the paramagnetic metal chelating label domains of the invention can comprise any suitable chelating moiety, or multiple chelating moieties. It will be appreciated that certain metals may be toxic in the absence of a chelator, such as Gd ion, and chelate forms will be preferred. In one aspect, the paramagnetic metals can be complexed with two or more chelators. A variety of suitable chelator moieties are known in the art, such as, for example, hydrazidonicotinamide (HYNIC), DRPA, EDTA, DOTA, TETA, DTPA and BAT. In another embodiment, the label domain is compatible with a nuclear imaging modality, such as, SPECT or PET, and can comprise a radionuclide such as, for example, ¹⁹⁹Au, ⁷²As, ¹⁴¹Ce, ⁶⁷Cu, ⁶⁰Cu, ⁵²Fe, ⁶⁷Ga, ⁶⁸Ga, ⁵¹Gr, ¹¹¹In, ¹⁷⁷Lu, ⁵¹Mn, ²⁰³Pb, ¹⁸⁸Re, ⁹⁷Ru, ⁴⁷Sc, ^{177m}Sn, ^{94m}Tc, ¹⁶⁷Tm, and ⁹⁰Y. The radionuclide can be chelated by a suitable chelator, or by multiple chelators, such as, for example HYNIC, DRPA, EDTA, DOTA, TETA, DTPA and BAT. Conditions under which a chelator will coordinate a metal are described, for example, by Gansow et al., U.S. Pat. Nos. 4,831,175, 4,454,106 and 4,472,509, each of which are incorporated herein by reference. In one embodiment, ^{99m}Tc is a particularly attractive radioisotope for therapeutic and diagnostic applications, as it is generally available to nuclear medicine departments, is inexpensive, gives minimal patient radiation doses, and has ideal nuclear imaging properties.

In embodiments wherein the novel imaging agents are compatible with MRI, the paramagnetic metal ions of the label domains can have atomic numbers 21-29, 42, 44, or 57-83. This includes ions of the transition metal or lanthanide series which have one, and more preferably five or more, unpaired electrons and a magnetic moment of at least 1.7 Bohr magneton. The preferred paramagnetic metal is selected from the group consisting of Gd(III), Fe(III), M(II and III), Cr(III), Cu(II), Dy(III), Tb(III), Ho(III), Er(III), and Eu(III). Gd(III) is particularly preferred for MRI due to its high relaxivity and low toxicity, and the availability of only one biologically accessible oxidation state. Gd(III) chelates have been used for clinical and radiologic MR applications since 1988, and approximately 30% of MR exams currently employ a gadolinium-based contrast agent.

The practitioner can select a metal according to dose required to detect a multidrug resistance phenotype and considering other factors such as toxicity of the metal to the subject. See, Tweedle et al., Magnetic Resonance Imaging (2nd ed.), vol. 1, Partain et al., eds. (W.B. Saunders Co. 1988), pp. 796-7, which is expressly incorporated herein by reference. Generally, the desired dose for an individual metal will be proportional to its relaxivity, modified by the biodistribution, pharmacokinetics and metabolism of the metal. The trivalent cation, Gd³⁺ is particularly preferred for the imaging agents of the invention, due to its high relaxivity and low toxicity, with the further advantage that it exists in only one biologically accessible oxidation state, which minimizes undesired metabolization of the metal by a patient. Another useful metal is Cr³⁺, which is relatively inexpensive.

The organic chelator of the label domain can be a molecule having one or more polar groups that act as a ligand for, and complex with, a paramagnetic metal or radionuclide, depending on which type of imaging modality is being used. Suitable chelators are known in the art and can include those listed above, as well as acids with methylene phosphonic acid groups, methylene carbohydroxamine acid groups, carboxyethylidene groups, or carboxymethylene groups. Examples of chelators include, but are not limited to, diethylenetriaminepentaacetic acid (DTPA), 1,4,7,10-tetraazacyclotetradecane-1,4,7,10-tetraacetic acid (DOTA), ethylenediaminetetraacetic acid (EDTA), and 1,4,8,11-tetraazacyclotetradecane-1,4,8,11-tetraacetic acid (TETA). Additional chelating ligands are ethylenebis-(2-hydroxy-phenylglycine) (EHPG), and derivatives thereof, including 5-Cl-EHPG, 5Br-EHPG, 5-Me-EHPG, 5t-Bu-EHPG, and 5sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl DTPA; bis-2 (hydroxybenzyl)-ethylene-diaminediacetic acid (HBED) and derivatives thereof, the class of macrocyclic compounds which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl) aminomethylbenzene (MECAM). A preferred chelator for use in the present invention is DOTA. Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. No. 4,899,755, all of which are hereby incorporated by reference. The skilled artisan will be able to select a suitable chelating moiety for a particular metal atom according to factors generally known in the art.

In general, the transduction and/or substrate domains of the compounds of the invention can be bound directly (by covalent or noncovalent interactions) to the metal chelate (or other detectable contrast agent), or it may be coupled or conjugated to the metal chelate using a linker moiety, which may be, without limitation, amide, urea, acetal, ketal, double ester, carbonyl, carbamate, thiourea, sulfone, thioester, ester, ether, disulfide, lactone, imine, phosphoryl, or phosphodiester linkages; substituted or unsubstituted saturated or unsaturated alkyl chains; linear, branched, or cyclic amino acid chains of a single amino acid or different amino acids (e.g., extensions of the N- or C-terminus of the fibrin binding moiety); derivatized or underivatized polyethylene glycol, polyoxyethylene, or polyvinylpyridine chains; substituted or unsubstituted polyamide chains; derivatized or underivatized polyamine, polyester, polyethylenimine, polyacrylate, poly(vinyl alcohol), polyglycerol, or oligosaccharide (e.g., dextran) chains; alternating block copolymers; malonic, succinic, glutaric, adipic and pimelic acids; caproic acid; simple diamines and dialcohols; and other simple polymeric linkers known in the art (see, e.g., WO 98/18497, WO 98/18496). The molecular weight of the linker moiety can be tightly controlled and no limitations should be placed on their structure with the exception that the linker moieties, if used, should not interfere with any of the multiple functions of the novel compounds, e.g. the translocation function, the detection function, and the MDR substrate function. The molecular weights can range in size from less than 100 g/mol to greater than 1000 g/mol. In addition, it may be desirable to utilize a linker that is biodegradable in vivo to provide efficient routes of excretion for the imaging compounds of the present invention. Depending on their location within the linker moiety, such biodegradable functionalities can include ester, double ester, amide, phosphoester, ether, acetal, and ketal functionalities. In certain embodiments, a linker can be an alkyl chain chaving from 2 to 6 carbon atoms in the chain, and may be substituted, e.g., with substituents to permit attachment of the linker to the two domains to be linked (e.g., the translocation domain and the imaging domain.

In general, known methods can be used to couple the metal chelate label domains together with the other components of the inventive compounds, i.e. the substrate and transduction domains, either directly or through linkers. See, e.g., WO 95/28967, WO 98/18496, WO 98/18497 and discussion therein. For example, a peptide moiety (e.g. transduction domain) can be linked through its N- or C-terminus via an amide bond, for example, to a metal coordinating backbone nitrogen of a chelating moiety, or to a functional group, such as an acetate group, of the metal chelating moiety. The present invention contemplates linking of the chelate on any position, provided the metal chelate retains the ability to bind the metal tightly. Similarly, the transduction and/or substrate domains may be modified or elongated (e.g. with a linker) in order to generate a locus for attachment to a metal chelate, provided such modification or elongation does not eliminate its ability to perform its function.

The novel imaging compounds of the invention can be prepared according to the disclosures herein and can be used in the same manner as conventional MRI contrast reagents. When imaging a cancer cell, certain MR techniques and pulse sequences may be preferred to enhance the contrast of the cancer cell or tissue to the background blood and tissues. These techniques include (but are not limited to), for example, black blood angiography sequences that seek to make blood dark, such as fast spin echo sequences (see, e.g., Alexander et al., Magnetic Resonance in Medicine, 40(2): 298-310 (1998)) and flow-spoiled gradient echo sequences (see, e.g., Edelman et al., Radiology, 177(1): 45-50 (1990)). These methods also include flow independent techniques that enhance the difference in contrast due to the T₁ difference of contrast-enhanced thrombus and blood and tissue, such as inversion-recovery prepared or saturation-recovery prepared sequences that will increase the contrast between thrombus and background tissues. In addition, since the present invention does not significantly alter T₂, methods of T₂ preparation may also prove useful (see, e.g., Gronas et al., Journal of Magnetic Resonance Imaging, 7(4): 637-643 (1997)). Finally, magnetization transfer preparations may also improve contrast with these agents (see, e.g., Goodrich et al., Investigative Radiology, 31(6): 323-32 (1996)).

The term “substrate domain” as used herein refers to a moiety capable of being transported from the cytoplasm of a cancer cell to the extracellular environment through a transporter associated with the multidrug resistance phenotype, e.g. an ATP binding cassette transporter, a multidrug resistance associated protein, MDR1, or P-glycoprotein. The particular transporter system can vary depending on the particular molecular and genetic characteristics of the multidrug resistance phenotype, e.g. multidrug resistance based on overexpression of p-glycoprotein. Accordingly, the substrate domain can be any known MDR transporter substrate, such as, a p-glycoprotein substrate, which becomes effluxed or “pumped” or transported from a cell's cytoplasm across the cellular membrane out into the extracellular environment when in contact with the MDR transporter. In one embodiment, the substrate domain is a rhodamine fluorescent dye, for example, carboxytetramethylrhodamine (TAMRA), which is a known substrate for p-glycoprotein (Twentyman et al., A comparison of rhodamine 123 accumulation and efflux in cells with p-glycoprotein-mediated and MRP-associated multidrug resistance phenotypes, Eur J. Cancer 30A, 1360-1369, 1994). The substrate domain can also be a hexakis (R-isonitrile) complex as disclosed in U.S. Pat. No. 5,403,574, which is incorporated herein by reference. In other embodiments, the substrate domain can be Hoechst 33342, daunorubicin or taxol, as disclosed in U.S. Pat. No. 6,861,431, which is hereby incorporated by reference.

In a particular embodiment, the present invention relates to an imaging compound wherein the transduction domain is the amphiphilic membrane translocation HIV-1 Tat basic domain peptide, the label domain is the GdDOTA chelate complex (an MR contrast agent), and the MDR substrate domain is carboxytetramethylrhodamine (TAMRA).

The present invention contemplates any suitable molecular arrangement of the domains of the imaging compounds of the invention so long as such configurations are consistent with the function of the inventive compounds, namely, the translocation of the compounds into a cell by the transduction domain, the transport out of a cancer cell having a multidrug resistance phenotype, including an MDR transporter, and the detection or imaging of the compound by detection or imaging of the label domain using a noninvasive means for imaging, especially a medical imaging modality, and especially MRI. For example, the transduction domain can be covalently or noncovalently bonded to the label domain and/or the substrate domain, either to each or only one of the domains, and either by direct or indirect linkage (e.g. through a linker moiety). Similarly, the label domain can be covalently or noncovalently bonded to the transduction domain and/or the substrate domain, either to each or only one of the domains, and either by direct or indirect linkage (e.g. through a linker moiety). Likewise, the substrate domain can be covalently or noncovalently bonded to the transduction domain and/or the substrate domain, either to each or only to one of the domains, and either by direct or indirect linkage (e.g. through a linker moiety). As used herein, the term “noncovalently bonded” is meant to be consistent with the known meaning in the art, namely, chemical interactions that include ionic interactions, van der Waals, hydrophobic interactions, and the like.

### Manufacture of Imaging Agents

The imaging agents and compounds of this invention can be prepared by a variety of methods, some of which are known in the art for preparation of imaging agents and the like.

For example, as described in the Examples, infra, a conjugate of HIV-1 Tat basic domain peptide (as a transduction domain), GdDOTA (a label moiety or domain) and carboxytetramethylrhodamine (an MDR substrate domain) can be prepared by derivatization of the peptidic transduction domain with a metal-chelating moiety (such as tetraazacyclododecanetetraacetic acid), e.g., by amide bond formation with a sidechain of a peptide residue of the peptidic transduction domain (in the Example, amide bond formation to a lysine side chain). For additional methods for preparing conjugates of a peptidic substrate with a metal chelator, see, e.g., U.S. Pat. No. 5,958,374, and references cited therein.

Similarly, the rhodamine-containing moiety can be covalently attached to the peptidic domain, e.g., using a linker such as an amino acid (e.g., a natural or unnatural amino acid or an alkyl amino acid such as 6-aminohexanoic acid), alkylene-diamine or alkylene-dicarboxylate linker, to secure the rhodamine moiety to the N-terminus of the peptidic domain. If desired, additional linker or spacer moieties can be used to provide appropriate chemical functionality. Protective groups can be used to prevent undesired reaction (e.g., of sidechain moieties) and removed when synthesis is complete (for examples of protective groups and conditions for their installation and removal, see, e.g., Greene and Wuts, “Protective Groups in Organic Synthesis”, 3rd ed., Wiley: New York, 1999).

Certain additional and/or overlapping aspects of the preparation of the compounds of the invention, e.g. preparing conjugates of transduction, label and substrate domains via linkers, is described herein elsewhere.

### Methods of Use

The present invention encompasses a variety of methods relating to the detection, imaging, monitoring, evaluation of, and diagnosis of a multidrug resistance phenotype in a cancer in a subject using the novel imaging compounds of the invention in combination with a noninvasive imaging modality, such as MRI or nuclear imaging. At a broad level, the imaging agents of the invention as described herein can be used in one or more of the following methods: (a) methods of detecting and/or imaging a multidrug resistance phenotype, (b) predictive methods (e.g. diagnostic methods, prognostic methods, monitoring clinical trials), (c) methods of treating (e.g. treating a cancer having a MDR phenotype or treating the MDR phenotype), and (d) methods of screening (e.g. inhibitors of MDR phenotype).

The invention is useful for detecting, treating and/or evaluating all types of cancers, including especially those cancers that have or will likely develop a multidrug resistance phenotype, including but not limited to, adrenal cancer, AIDS-related lymphoma, anal cancer, ataxia-telangiectasia, bladder cancer, brain tumors, breast cancer, cervical cancer, chronic lymphocytic leukemia, chronic myelogenous leukemia, colorectal cancer, craniopharyngioma, cutaneous T-cell lymphoma, endometrial and uterine cancer, esophageal cancer, Ewing's sarcoma, fallopian tube cancer, gallbladder cancer, gastric cancer, gestational trophoblastic disease, choriocarcinoma, Hairy cell leukemia, Hodgkin's disease, Kaposi's sarcoma, kidney cancer, laryngeal cancer, leukemia including acute lymphocytic leukemia and acute myelogenous leukemia, Li-Fraumeni syndrome, liver cancer, lung cancer, Hodgkin's lymphoma, Non-Hodgkin's lymphoma, medulloblastoma, melanoma, mesothelioma, metastases, myelomas, myeloproliferative disorders, neuroblastoma, Non-Hodgkin's disease, non-small cell lung dancer, oropharyngeal cancers, osteosarcoma, ovarian cancer, pancreatic cancer, parathyroid cancer, penile cancer, pituitary cancer, prostate cancer, retinoblastoma, rhabdomyosarcoma and other soft-tissue sarcomas, osteosarcoma, small intestine cancer, small-cell lung cancer, testicular cancer, thymoma, thyroid cancer, urethal cancer, vaginal cancer, vulvar cancer and Wilms' tumor.

The term “cancer” for the purposes of this invention is meant to encompass any type and/or stage of cancer, from pre-cancerous cells and/or tissues to benign and/or malignant cancers to solid tumors or circulating cancers. The term further includes malignancies characterized by deregulated or uncontrolled cell growth, for instance carcinomas, sarcomas, leukemias, and lymphomas. The term “cancer” includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor. The term “cancer” further encompasses any additional terminology used in the art to refer to cancer. For example, the term cancer encompasses a “carcinoma.” The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. The term “adenocarcinoma” includes carcinomas derived from glandular tissue or a tumor in which the tumor cells form recognizable glandular structures. The term “sarcoma” is also encompassed by the term “cancer” and includes malignant tumors of mesodermal connective tissue, e.g., tumors of bone, fat, and cartilage.

The term “cancer” can also refer to a neoplasm. The term “neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli that evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histology and other features of a neoplasm, it can be determined whether the neoplasm is benign or malignant. Invasion and metastasis (the spread of the neoplasm to distant sites) are definitive attributes of malignancy.

Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. In contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue, may be converted to a fibrous connective tissue capsule allowing for easy surgical removal of the benign tumor.

Conversely, malignant tumors are locally invasive and grow into the adjacent tissues usually giving rise to irregular margins that are not encapsulated making it necessary to remove a wide margin of normal tissue for the surgical removal of malignant tumors. Benign neoplasms tend to grow more slowly and tend to be less autonomous than malignant tumors. Benign neoplasms tend to closely histologically resemble the tissue from which they originated. More highly differentiated cancers, i.e., cancers that resemble the tissue from which they originated, tend to have a better prognosis than poorly differentiated cancers, while malignant tumors are more likely than benign tumors to have an aberrant function, e.g., the secretion of abnormal or excessive quantities of hormones.

Accordingly, the imaging agents and methods of using the imaging agents of the invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary-carcinoma, transitional cell carcinoma, chorioaencinoma, semonoma, or embryonal carcinoma; and tumors of the nervous system including gioma, menigoma, medulloblastoma, schwannoma or epidymoma, in order to monitor, detect, diagnose, image and/or evaluate the cancer for a multidrug resistance phenotype, especially during, in anticipation of, or after a treatment by one or more chemotherapeutic agents. Additional cell types amenable to treatment and/or diagnosis according to the methods described herein include those giving rise to mammary carcinomas, gastrointestinal carcinoma, such as colonic carcinomas, bladder carcinoma, prostate carcinoma, and squamous cell carcinoma of the neck and head region.

Typical subjects to which compounds of the invention may be administered and to which the methods of the invention may be practiced will be mammals, particularly primates, especially humans, and especially those mammals having a cancer, especially, cancer having a multidrug resistance phenotype. For veterinary applications, a wide variety of subjects will be suitable, e.g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications, especially such sample having or containing or comprising cancer, in particular, cancer having a multidrug resistance phenotype.

A. Detection and/or Imaging Methods

In one aspect, the present invention provides a method for detecting a cancer cell having a multidrug resistance phenotype comprising providing an imaging agent as described herein, contacting the cancer cell with the imaging agent, and making an image using a medical imaging modality, wherein the imaging agent comprises a transduction domain capable of translocating the agent into the cancer cell, a label domain capable of being detected by the medical imaging modality, and a substrate domain capable of being transported by a multidrug resistance transporter.

In another aspect, the present invention relates to an imaging method comprising the steps of: providing an imaging compound according to Formula 1:

[Y¹—Y³]—Y²

wherein, Y¹ is a transduction domain; Y² is a contrast agent or “label domain” capable of being detected by an imaging modality; Y³ is a multidrug resistance transporter substrate or “substrate domain”; contacting cells or tissues with the compound; and making an image with an imaging modality.

In certain embodiments, the detection methods of the invention can be performed in vitro. In such embodiments, the cancer cell or tissue can be from any suitable source, such as for example, a biopsy or a cell or tissue culture. Methods for obtaining biopsies and maintaining and/or propagating the removed tissues and/or cells will be well known to the skilled artisan. In vitro detection of multidrug resistance can have various applications, such as, for example, determining whether a particular subject's cancer, either before, during or after treatment, has developed a multidrug phenotype. Such applications also can include the use of the compounds of the invention to screen for inhibitors of the MDR phenotype, e.g. screening for inhibitors an MDR transporter such as P-glycoprotein.

The type of imaging modality used to detect the compounds of the invention will depend on the particular label domain used in the inventive compounds. For example, if the label domain comprises a gadolinium chelate, then typically MRI could be used to detect the imaging agent of the invention. If a radionuclide chelate is used as the label domain, a nuclear imaging method could be used. If a fluorescence-based label domain is used, an optical imaging system could be used, such as, for example a FACS system or fluorescence microscopy or a fluorescence automated plate reader. Choosing an appropriate imaging modality for use in the in vitro detection methods of the invention are completely within the knowledge of the skilled artisan.

In addition, the amount of imaging agent used in the in vitro detection methods of the invention will be determined by one of ordinary skill in the art and can depend on the degree to which the MDR phenotype is present, e.g. the level of expression of the MDR transport system (e.g. the P-glycoprotein). The skilled artisan can determine what amount of the novel imaging compounds that is sufficient for detecting a MDR phenotype without undue experimentation, i.e. a detectably sufficient amount.

In other embodiments, the methods of detection take place in vivo. As noted above, the type of cancer in which an MDR phenotype can be detected is not limited to any particular type and can broadly include any solid or circulating tumor.

The type of imaging modality for the detection of the MDR phenotype is not limited to any particular type, and can include, for example MRI, nuclear imaging (e.g. PET or SPECT), optical imaging, sonoluminence imaging or photoacoustic imaging (ultrasound). The skilled artisan will appreciate that the particular label domain of the imaging compounds of the invention should be compatible with the particular imaging modality being used.

In a preferred embodiment, the methods of detection utilize imaging agents that are capable of being detected by MRI. For example, the imaging agent can comprise a label domain that is a MR contrast agent, such as, for example a paramagnetic metal chelate or chelates or any of those described herein. The imaging agent can also comprise a radionuclide label domain for imaging or detecting by a nuclear imaging modality, such as, positron emission tomography (PET) or single photon emission computer tomography (SPECT).

As mentioned herein elsewhere, the skilled artisan will be capable of determine the particular amount (and route of administration, etc.) of the novel imaging compounds such that a detectably effective amount, i.e. an amount that is sufficient to detect or image the MDR phenotype. Such a determination will of course take into account any toxicity issues relating to the administration of the compound, and any other relevant health considerations such that the subject does not become harmed upon receiving the imaging compounds.

In a particular embodiment, the detection methods using the compounds of the invention are carried before the subject is administered any chemotherapeutic therapy against a cancer. It will be appreciated that pre-treatment detection of MDR would be useful to detect MDR that spontaneously arises in the absence of a chemotherapeutic treatment. In other embodiments, the present detection methods can be carried during an ongoing chemotherapeutic treatment or after the completion of a chemotherapeutic treatment in order to detect, monitor and/or evaluate the progression and/or incidence of a MDR phenotype in connection with a therapy.

In one aspect, the present invention relates to a method of detecting and/or evaluating and/or imaging a multidrug phenotype in a subject undergoing or having undergone a anticancer treatment whereby the subject is administered (at the same time or substantially the same time, e.g. co-administered) a compound of the invention or a pharmaceutical composition of the invention as described elsewhere herein in a detectably sufficient amount. A medical imaging modality, which is compatible with the particular label domain, is then administered to the subject to obtain or make an image of the compound which in turn provides an image of the cells or tissues targeted by the imaging compounds of the invention.

In certain embodiments, the detection and/or imaging methods of the invention can be carried out in connection with a an inhibitor or “reversing agent” which can assist in distinguishing multidrug resistant cells from non-resistant cells in a equivalent manner as that disclosed in U.S. Pat. No. 5,403,574, which is herein incorporated by reference. In addition, any suitable control is contemplated by the present invention which will assist in the detecting and determination of a multidrug resistance phenotype, such as, for example, comparing an image obtained from a subject having an MDR cancer to an image obtained from a subject not having an MDR cancer. For example, in one aspect, the net cellular accumulation of an agent of the invention administered alone can be compared with the net cellular accumulation of the agent with it is co-administered with an agent that inhibits the MDR phenotype. In the presence of the inhibitor, the agent is not excluded from the cells, whereas in the absence of an inhibitor, exclusion of the agent from the cell is apparent. Typical inhibitors are described herein elsewhere and can include, but are not limited to, verapamil, quinidine, vinblastine, vincristine, adriamycine, colchicine, daunomycin, cactinomycin, vanadate, cyclosporine and tetraphenylborate. The use of such inhibitors and controls in the detection methods of the invention and the advantages thereof will be appreciated by and are within the abilities of a person of ordinary skill in the art.

In one embodiment of the invention, a subject receives an imaging agent of the invention in both the presence and absence of an MDR inhibitor. The treatment is in either order. If the two drugs are first administered together, then following the detection process, the inhibitor is given sufficient time to leave the system before the administration of the imaging agent alone. Following the treatments and detection, the measurements of accumulation of the imaging agent in both cases are compared. Multidrug resistance tissue is detected in the presence of an inhibitor but not in its absence. Using this method, multidrug resistant tissue is located without invasive procedures.

The methods of the present invention are also applicable to whole tissue and cells in vitro. The invention is advantageous over current methods of determining the multidrug resistance phenotype in vitro because it is rapid and simple. Using presently available methods, before the multidrug resistance phenotype can be evaluated in whole tissue, a single cell suspension must be created (e.g., for flow cytometry) or even more laborious techniques must be used, such as monolayer cell culture. Using the method of the current invention, it is possible to detect the multidrug resistance phenotype in tissue without, or with minimum, disaggregation. Thus, therapeutic regimens may be decided with less delay than with presently available methods.

Current in vitro procedures for detecting the multidrug resistance phenotype involve forming either cell monolayers or single cell suspensions because the detectable emission (i.e., beta rays or fluorescence) does not penetrate and pass through intervening biological material. Thus, there is no rapid procedure for assaying the multidrug resistance phenotype of cells in a tissue or a cell mass, such as a tumor or tumor biopsy. Tissue would have to be dispersed into single cells for analysis and may have to be cultured. Cell culture, however is time consuming and also alters the selection pressures so that the cultured cells do not display the same phenotype or genotype as the cells in vivo. For example, the overexpression of the multidrug resistance gene in a tumor occurs as a result of the selection and multiplication of single or a few mutant cells as the tumor is subjected to a chemotherapeutic drug. If the tumor is excised and grown in tissue culture, the genotype may change because the selection pressure is not the same. This may interfere with the proper analysis of the tumor and hence with prescription of a effective therapeutic regimen. With the methods of the present invention, however, the tumor could be analyzed without dispersion and growth in culture. Relevant prescription would then be more likely.

Further, tumors are usually genotypically and phenotypically heterogeneous. New genotypes may arise in a very small or minute portions of a tumor and may not be detectable by routine methods. For example, the multidrug resistance phenotype occurring in a small area of a tumor, may be missed if the tumor cells are dispersed or merely biopsied. With the methods of the present invention, since a small area would be intact, imaging the tumor would reveal such small pockets of multidrug resistant cells.

Accordingly, the invention embodies methods of assaying the multidrug resistance phenotype in whole tissue or tissue biopsies by incubating the tissue or biopsy with the imaging agents of the present invention. In a preferred embodiment, the tissue is exposed to the imaging agent in the presence and absence of an MDR inhibitor, such as those mentioned above. Accumulation of the imaging agent in the tissue is measured in both cases and the measurements are compared. In alternative embodiments, the imaging agent is administered alone and the measurement obtained is compared with the measurement obtained with normal control tissue. In one preferred embodiment, the agent Tat-GdDOTA-TAMRA. Alternative imaging agents include, but are not limited to the agents described herein above.

Accordingly, the methods of detection of the invention as described herein above embody detecting MDR phenotypes in tissues and/or subjects, in in vitro or in vivo settings, without the need for invasive procedures and in a manner which is advantageously efficient, rapid and effective.

B. Predictive Methods

The present invention further relates to predictive methods such as diagnostic methods for diagnosing a multidrug resistance phenotype in a subject. The predictive methods of the invention also include prognostic methods which probe whether a subject is at risk for developing a condition associated with a multidrug resistance phenotype, whether a particular chemotherapeutic treatment is or may be suitable for a given cancer, or whether a particular chemotherapeutic treatment should be modified or changed or optimized.

In one embodiment, the present invention provides a method for diagnosing a multidrug resistance phenotype in a subject comprising providing an imaging agent as described herein, contacting a cancer cell with the imaging agent, and making an image using a medical imaging modality, wherein the imaging agent comprises a transduction domain capable of translocating the agent into the cancer cell, a label domain capable of being detected by the medical imaging modality, and a substrate domain capable of being transported by a multidrug resistance transporter. The imaging agent is administered in a detectably effective amount which is sufficient to detect the MDR phenotype. The subject can be tested before, during or after a treatment using a chemotherapeutic agent.

In another embodiment, the effectiveness of a treatment with a chemotherapeutic agent can be evaluated by a method comprising administering to the subject an imaging agent in an amount sufficient to detect in the subject a multidrug resistance phenotype if present, making an image using a medical imaging modality, reading the image to detect a multidrug resistance phenotype if present, wherein a lack of detected multidrug resistance phenotype predicts an effective treatment with the chemotherapeutic agent, and wherein the imaging agent comprises a transduction domain capable of translocating the agent into the cancer cell, a label domain capable of being detected by the medical imaging modality, and a substrate domain capable of being transported by a multidrug resistance transporter.

With the method of the present invention, it is also possible to evaluate in vivo the efficacy of alternative chemotherapeutic drugs. In one embodiment of the invention, the ability of a drug to act as a chemosensitizer (reversing agent or inhibitor used in chemotherapy to facilitate the uptake of a chemotherapeutic drug in drug-resistant tumor cells) is determined. An imaging agent of the present invention and a potential chemosensitizer are administered to a patient. If the drug is able to reverse the multidrug resistance phenotype, the agent will be retained in the patient's tumor cells in the presence of that drug but not in its absence. The chemosensitizer can then be used to facilitate the administration of or to test the efficacy of anti-tumor drugs.

The diagnostic methods described herein can furthermore be utilized to identify subjects having or at risk of developing a disease or disorder associated with aberrant or unwanted MDR transporter expression or activity, e.g. P-glycoprotein overexpression. As used herein, the term “aberrant” includes an MDR transporter expression or activity which deviates from the wild type MDR transporter expression or activity. Aberrant expression or activity includes increased or decreased expression or activity, as well as expression or activity which does not follow the wild type developmental pattern of expression or the subcellular pattern of expression. For example, aberrant MDR transporter expression or activity is intended to include the cases in which a mutation in the MDR transporter gene (or genetic regulatory sequences) causes the MDR transporter gene to be under-expressed or over-expressed and situations in which such mutations result in a non-functional MDR transporter protein or a protein which does not function in a wild-type fashion. As used herein, the term “unwanted” includes an unwanted phenomenon involved in a biological response. For example, the term unwanted includes an MDR transporter, e.g. P-glycoprotein, expression or activity which is undesirable in a subject.

The assays described herein, such as the preceding diagnostic assays or the following assays, can be utilized to identify a subject having or at risk of developing a disorder associated with a misregulation in MDR transporter protein activity or nucleic acid expression. Alternatively, the prognostic assays can be utilized to identify a subject having or at risk for developing a disorder associated with a misregulation in MDR transporter protein activity or nucleic acid expression. Thus, the present invention provides a method for identifying a disease or disorder associated with aberrant or unwanted MDR transporter expression or activity in which a test sample is obtained from a subject and MDR transporter protein is detected, wherein the presence of MDR transporter protein is diagnostic for a subject having or at risk of developing a disease or disorder associated with aberrant or unwanted MDR transporter expression or activity.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted MDR transporter expression or activity, e.g., a cancer where the cells of the cancer have developed multidrug resistance. Thus, the present invention provides methods for determining whether a subject can be effectively treated with an agent for a disorder associated with aberrant or unwanted MDR transporter expression or activity in which a test sample is obtained and MDR transporter protein of the MDR phenotype is detected (e.g., wherein the abundance of MDR transporter protein or activity is diagnostic for a subject that can be administered the agent to treat a disorder associated with aberrant or unwanted MDR transporter expression or activity).

Monitoring the influence of agents (e.g., drugs) on the expression or activity of an MDR transporter protein can be applied not only in MDR transporter drug screening, but also in clinical trials. The effectiveness of an agent determined by a screening assay to decrease MDR transporter gene expression, protein levels, or downregulate MDR transporter activity, can be monitored in clinical trials of subjects exhibiting increased MDR transporter gene expression, protein levels, or upregulated MDR transporter activity. In such clinical trials, the expression or activity of an MDR transporter gene, and preferably, other genes that have been implicated in, for example, an MDR-associated disorder can be used as a “read out” or markers of the phenotype of a particular cell.

In one embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an MDR transporter protein in the preadministration sample; (iii) obtaining one or more post-administration samples from the subject; (iv) detecting the level of expression or activity of the MDR transporter protein in the post-administration samples; (v) comparing the level of expression or activity of the MDR transporter protein in the pre-administration sample with the MDR transporter protein in the post administration sample or samples; and (vi) altering the administration of the agent to the subject accordingly. For example, increased administration of the agent may be desirable to increase the expression or activity of MDR transporter to higher levels than detected, i.e., to increase the effectiveness of the agent. Alternatively, decreased administration of the agent may be desirable to decrease expression or activity of MDR transporter to lower levels than detected, i.e. to decrease the effectiveness of the agent. According to such an embodiment, MDR transporter expression or activity may be used as an indicator of the effectiveness of an agent, even in the absence of an observable phenotypic response.

C. Treatment Methods

In another aspect, the present invention provides a method for preventing or reducing multidrug resistance cancer in a subject undergoing treatment with a chemotherapeutic agent comprising administering to the subject an imaging agent in an amount sufficient to detect in the subject a multidrug resistance phenotype if present, making an image using a medical imaging modality, reading the image to detect a multidrug resistance phenotype if present, and optimizing the chemotherapeutic treatment if a multidrug resistance phenotype is detected thereby preventing or reducing the multidrug resistant cancer in the subject, wherein the imaging agent comprises a transduction domain capable of translocating the agent into the cancer cell, a label domain capable of being detected by the medical imaging modality, and a substrate domain capable of being transported by a multidrug resistance transporter. The step of optimizing the chemotherapeutic treatment can comprise administering an inhibitor of the multidrug resistance phenotype, adjusting the treatment with a chemotherapeutic agent such that it becomes effective against the multidrug resistance phenotype, or administering a different chemotherapeutic agent known to be effective against the multidrug resistance phenotype. Possible inhibitors are described herein elsewhere.

The invention also embodies methods of designing chemotherapy regimens by assaying the multidrug resistance phenotype in patients or their explanted tissue either prior to or during treatment. During the course of chemotherapy, when it is determined that a multidrug resistance-negative tumor (previously showing agent localization) converts or recurs with multidrug resistance (expressed as loss of agent localization), this valuable information is used to guide therapeutic management of the patients. Accordingly, in an embodiment of the invention, patients are evaluated for the multidrug resistance phenotype prior to initiation or continuation of chemotherapy. Those patients deemed phenotypically multidrug resistance-positive are spared the toxic and debilitating side effects of futile chemotherapy and alternative regiments or treatment ought.

Further, the location of tumors not detectable by standard means (e.g., CAT scan) is determinable if these tumors have the multidrug resistance phenotype. Thus, the methods of the present invention provide means to monitor progression or regression of the disease during chemotherapy.

D. Screening Methods

In another aspect, the present invention provides a method for screening potential inhibitors of a multidrug resistance phenotype comprising providing an imaging agent as described herein, contacting an MDR cancer cell with the imaging agent in the presence and absence of a potential inhibitor, and making an image using a medical imaging modality, wherein the imaging agent comprises a transduction domain capable of translocating the agent into the cancer cell, a label domain capable of being detected by the medical imaging modality, and a substrate domain capable of being transported by a multidrug resistance transporter, and wherein the detection of the imaging agent in the MDR cancer cell indicates an inhibitor of the MDR phenotype.

In the screening assay embodiments of the invention, candidate or test compounds which bind to or modulate the activity of or inhibit an MDR transporter protein or polypeptide or biologically active portion thereof, such as, for example, P-glycoprotein translocator or the MDR associated 190 kDa protein translocator. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310).

In one embodiment, an assay is a cell-based assay in which a cell which expresses an MDR translocator or biologically active portion thereof, i.e. an MDR phenotype, is contacted with a test compound and the ability of the test compound to modulate the MDR phenotype is determined. Determining the ability of the test compound to modulate the MDR phenotype can be accomplished by monitoring, for example, cellular transport of the imaging agent.

In one embodiment, suitable test compounds include, but are not limited to, verapamil, desmethoxyverapamil, chloroquine, quinine, chinchonidine, primaquine, tamoxifen, dihydrocyclosporin, yohimbine, corynanthine, reserpine, physostigmine, acridine, acridine orange, quinacrine, trifluoroperazine chlorpromazine, propanolol, atropine, tryptamine, forskolin, 1,9-dideoxyforskolin, cyclosporin, (U.S. Pat. No. 4,117,118 (1978)), PSC-833 (cyclosporin D, 6-[(2S,4R,6E)-4-methyl-2-(methylamino)-3-oxo-6-octenoic acid]-(9CI) [U.S. Pat. No. 5,525,590] [ACS 121584-18-7], Keller et al., “SDZ PSC 833, a non-immunosuppressive cyclosporine: its potency in overcoming P-glycoprotein-mediated multidrug resistance of murine leukemia”, Int J Cancer 50:593-597 (1992)), RU-486 (17β-hydroxy-11β-[4-dimethylaminophenyl]-17αprop-1-ynyl estra-4,9-dien-3 one), RU-49953 (17β-hydroxy-11β,17α-[4-dimethylaminophenyl]-17βprop-1-ynyl estra-4,9 dien-3 one), S9778 (6-{4-[2,2-di( )-ethylamino]-1-piperidinyl}-N,N′, di-2-propenyl-1,3,5-triazine-2,4-diamine, bismethane sulfonate, [U.S. Pat. No. 5,225,411; EP 466586] [ACS #140945-01-3]; Dhainaut et al., “New triazine derivatives as potent modulators of multidrug resistance,” J Medicinal Chemistry 35:2481-2496 (1992)), MS-209 (5-[3-[4-(2,2-diphenylacetyl)piperazin-1-yl]-2-hydroxypropoxy]quinoline sesquifumarate, [U.S. Pat. No. 5,405,843 (continuation of U.S. Pat. No. 5,112,817)], [ACS #158681-49-3], Sato et al., “Reversal of multidrug resistance by a novel quinoline derivative, MS-209, Cancer Chemother Pharmacol 35:271-277 (1995)), MS-073 (Fukazawa et al., European Patent Application 0363212 (1989)), FK-506 (Tanaka et al., M. Physicochemical properties of FK-506, a novel immunosuppressant isolated from Streptomyces tsukubaensis” Transplantation Proceedings. 19(5 Suppl 6): 11-6, (1987); Naito et al., “Reversal of multidrug resistance by an immunosuppressive agent FK-506,” Cancer Chemother & Pharmacol. 29:195-200 (1992); Pourtier-Manzanedo et al., “FK-506 (fujimycin) reverses the multidrug resistance of tumor cells in vitro,” Anti-Cancer Drugs 2:279-83 (1991); Epand & Epand, “The new potent immunosuppressant FK-506 reverses multidrug resistance in Chinese hamster ovary cells,” Anti-Cancer Drug Design 6:189-93 (1991)), VX-710 (2-peperidinecarboxylic acid, 1-[oxo(3,4,5-trimethoxyphenyl)acetyl]-3-(3-pyridinyl)-1-[3-(3-pyridinyl)propyl]butyl ester [ACS 159997-94-1] [U.S. Pat. No. 5,620,971] Germann et al., “Chemosensitization and drug accumulation effects of VX-710, verapamil, cyclosporin A, MS-209 and GF120918 in multidrug resistance-associated protein MRP” Anti-Cancer Drugs 8, 141-155 (1997); Germann et al., “Cellular and biochemical characterization of VX-710 as a chemosensitizer: reversal of P-glycoprotein-mediated multidrug resistance in vitro” Anti-Cancer Drugs 8, 125-140 (1997)), VX-853 ([U.S. Pat. No. 5,543,423] [ACS #190454-58-1), AHC-52 (methyl 2-(N-benzyl-N-methylamino)ethyl-2,6-dimethyl-4-(2-isopropylpyrazolo[1,5-a]pyridine-3-yl)-1,4-dihydropyridine-3,5-dicarboxylate; [Japanese Patent 63-135381; European Patent 0270926] [ACS 119666-09-0] Shinoda et al., “In vivo circumvention of vincristine resistance in mice with P388 leukemia using a novel compound, AHC-52,” Cancer Res-49:1722-6 (1989)), GF-120918 (9,10-dihydro-5-methoxy-9-oxo-N-[4-[2-(1,2,3,4-tetrahydro-6,7-dimethoxyiso quinol-2-yl)ethyl]phenyl]-4 acridinecarboxamide, [U.S. Pat. No. 5,604,237] [ACS #143664-11-3] et al., “In vitro and in vivo reversal of multidrug resistance by GF 120918, an acridonecarboxamide derivative,” Cancer Res 53:4595-4602 (1993)), and XR-9051 (3-[(3Z,6Z)-6-Benzylidene-1-methyl-2,5-dioxopiperazin-3-ylidenemethyl]-N-[4-[2-(6,7-dimethoxy-1,2,3,4-tetrahydroisoquinolin-2-yl)ethyl]phenyl]benzamide hydrochloride, [ACS #57-22-7]).

The screening methods of the invention can be used with any of the imaging agents described herein. The present invention contemplates the screening of any number of compounds by manual or automated means using any suitable technology available in the art for high throughput screening methods, such as, for example, microtiter plates or microwell arrays. The imaging agent can be administered in the screening assays of the invention in an amount sufficient to allow for detection of a multidrug phenotype, i.e. a detectably effective amount. The skilled artisan will readily be able to ascertain what amount of the imaging agent to use in any given assay. The skilled artisan will also be able to determine which particular imaging modality will be appropriate for a given screen based in part on the type imaging agent being used. The MDR cancer cell can be obtained by any suitable means, for example, from a biopsy of a subject having an MDR phenotype. The skilled artisan will appreciate how to obtain the cells, manipulate and maintain the cells, culture the cells, and carry out the screening methods of the invention without undue experimentation.

### Pharmaceutical Compositions and Formulations and Dosages

The invention provides for pharmaceutical compositions and formulations comprising an agent for imaging a multidrug resistance phenotype and a pharmaceutically acceptable carrier. In addition to the active ingredients, e.g. the novel imaging agents disclosed herein or pharmaceutically acceptable salts thereof, these pharmaceutical compositions may contain a suitable pharmaceutically acceptable carrier and can be used pharmaceutically, e.g. for administration to a subject having a multidrug resistant cancer for the purpose of, for example, the monitoring, evaluation, detection or imaging of the multidrug resistance phenotype using a noninvasive imaging modality.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Administration of the pharmaceutical compositions of the invention can be by any suitable means, such as, for example oral administration, parenteral administration, transdermal administration, nasal administration, topical administration or by direct injection into or substantial nearby a cancer to be characterized or evaluated with respect to its multidrug resistance features. Administration of the pharmaceutical compositions of the invention can also be carried at or substantially at the same time as the administration of one or more chemotherapeutic agents, i.e. during or substantially at the same time as a chemotherapeutic treatment or anticancer treatment. The imaging agents of the invention and the one or more chemotherapeutic agents can be formulated as a single pharmaceutical composition or prepared as separate compositions. The amount of active ingredient that can be combined with a carrier material to produce a single dosage form will generally be that amount of the compound that enables the detection of a multidrug resistance phenotype by the methods of the invention. Generally, out of one hundred percent, this amount will range from about 1 percent to about ninety-nine percent of active ingredient, preferably from about 5 percent to about 70 percent, most preferably from about 10 percent to about 30 percent.

Pharmaceutical compositions for oral administration can be formulated using pharmaceutically acceptable carriers well known in the art in dosages suitable for oral administration and which are in such an amount or dosage which is sufficient to detect a multidrug resistance phenotype. Such carriers enable the pharmaceutical compositions to be formulated as tablets, pills, dragees, capsules, caplets, liquids, gels, gel caps, syrups, slurries, suspensions and the like, for ingestion by the subject.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. Injection of the compositions of the invention can be carried out by directly injecting the compositions into or substantially nearby a tumor or solid cancer.

Compounds of the invention can also be delivered directly to selected sites in the body, e.g. a tumor site, by a variety of means, including injection, infusion, catheterization and topical application, among others. Compounds of the invention also may be bound to carrier bio-compatible particles, e.g., autologous, allogenic or zenogenic cells, to facilitate targeted delivery of the substance. Unless otherwise specified, the discussion set forth below refers to binding of compounds of the invention to cells, either by direct delivery to the disease site, or in the preparation of carrier vehicles.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

For nasal administration, penetrants appropriate to the particular barrier to be permeated can be used in the formulation. Such penetrants are generally known in the art.

The pharmaceutical compositions of the present invention may be manufactured in a manner known in the art e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the compounds of the invention from subcutaneous or intramuscular injection. This may be accomplished by the use of a liquid suspension of crystalline or amorphous material having poor water solubility. The rate of absorption of the compounds then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The present invention further contemplates the co-administration of an imaging agent of the invention, or a pharmaceutical composition thereof, together with one or more anticancer compounds. The imaging agent can be administered, in particular, to monitor, detect or evaluate a multidrug resistance phenotype before, during or after treatment with the anticancer compound. For the purposes of this invention, “co-administering” is administration of two or more compounds, or pharmaceutical compositions comprising the compounds at the same time or at about the same time, e.g. sequential administration. Sequential administration also encompasses an administration regimen occurring in some pattern over the course of days, weeks, or months, such as, for example, administering on a first day an imaging agent or composition thereof followed by on a second day an anticancer treatment. There is no intended limitation as to the exact manner by which co-administration may occur and the skilled artisan will be able to competently design a suitable co-administration regimen comprising the imaging agents of the invention and one or more anticancer compounds.

The anticancer compounds contemplated by the present invention are limitless and include any of those known in the art, and especially include those anticancer compounds which are susceptible to the multidrug resistance phenotype, e.g. those compounds which are recognized by an MDR transporter and efflux pumped out of the cancer cell. Exemplary cancer therapeutic agents include, but are not limited to, chemical or biological reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990) and Teicher, B. A. Cancer Therapeutics: Experimental and Clinical Agents (1996) Humana Press, Totowa, N.J.), and are typically used to treat neoplastic diseases. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

The pharmaceutical compositions of the invention can also include additional compounds to control or treat infections that may be arise in parallel to the cancer under treatment. Such additional compounds can be formulated together with the imaging agents of the invention, or formulated separately alone or in combination with other active ingredients, such as the above mentioned anticancer compounds. The compounds for treating infections associated with cancer can include any known anti-viral, anti-fungal, anti-parasitic, or anti-bacterial compound that is compatible with the imaging agents and methods of the invention and given in dosages that are safe and effective. For example, the compounds can be anti-bacterial drugs. Anti-bacterial antibiotic drugs are well known and can include: penicillin Q, penicillin V, ampicillin, amoxicillin, bacampicillin, cyclacillin, epicillin, hetacillin, pivampicillin, methicillin, nafcillin, oxacilln, cloxacillin, dicloxacillin, flucloxacillin, carbenicillin, ticarcillin, avlocillin, mezlocillin, piperacillin, amdinocillin, cephalexin, cephradine, cefadoxil, cefaclor, cefazolin, cefuroxime axetil, cefamandole, cefonicid, cefoxitin, cefotaxime, ceftizoxime, cefinenoxine, ceftriaxone, moxalactam, cefotetan, cefoperazone, ceftazidme, imipenem, clavulanate, timentin, sulbactam, neomycin, erythromycin, metronidazole, chloramphenicol, clindamycin, lincomycin, vancomycin, trimethoprim-sulfamethoxazole, aminoglycosides, quinolones, tetracyclines and rifampin and antibacterial antibodies. (See Goodman and Gilman's, Pharmacological Basics of Therapeutics, 8th Ed., 1993, McGraw Hill Inc.).

The pharmaceutical compositions of the invention can additionally include compounds which act to inhibit the MDR phenotype and/or conditions associated with MDR phenotype. Such compounds can include any known MDR inhibitor compounds in the art, such as, antibodies specific for MDR components (e.g. anti-MDR transporter antibodies) or small molecule inhibitors of MDR transporters, including specifically, tamoxifen, verapamil and cyclosporin A, which are agents known to reverse or inhibit multidrug resistance. (Lavie et al. J. Biol. Chem. 271: 19530-10536, 1996, incorporated herein by reference). Such compounds can be found in U.S. Pat. Nos. 5,773,280, 6,225,325, and 5,403,574, each of which are incorporated herein by reference. Such MDR inhibitor compounds can be co-administered with the imaging agents of the invention for various purposes, including, reversing the MDR phenotype following the detection of the MDR phenotype to assist or enhance a chemotherapeutic treatment. The MDR inhibitor, such as, for example, tamoxifen, verapamil or cyclosporin A, may be used in conduction with the imaging compounds of the invention to assist in the detection of the MDR phenotype. In accordance with this aspect, an MDR inhibitor can enhance the uptake and accumulation of an imaging compound of the invention in an MDR cancer cell since the capacity of the MDR transport system in transporting or “pumping out” the imaging compound vis-a-vis the substrate domain would be diminished in the presence of an MDR inhibitor. Imaging in accordance with the methods described herein of the imaging compounds in the presence versus the absence of an MDR inhibitor and the comparison thereof could facilitate the detection of an MDR phenotype.

After pharmaceutical compositions comprising a compound of the invention formulated in an acceptable carrier have been prepared, they can be placed in an appropriate container and labeled for use in accordance with the methods described herein along with information including amount, frequency and method of administration and methods for imaging the MDR phenotype using the administered compounds.

The pharmaceutical composition may be formulated from a range of preferred doses, as necessitated by the condition of the patient being treated. For example, the imaging compounds described herein may preferably be 60%, 61%, 62%, 63%, 64%, 65%, 70%, 75%, 80%, 85%, 86%, 87%, 88%, 89%, 90%, and any percentage between 60% and 90%, of the weight of the composition.

In embodiments involving the co-administration of another active ingredient, such as, for example, an anticancer agent or an anti-bacterial agent, the imaging agents of the invention can be administered in combination therewith in a ratio in the range of 1:1-1:5, 1:1-1:10, 1:1-1:25, 1:1-1:50. 1:1-1:100, 1:1-1:500, 1:1-1:1000, 1:1-1:10,000, 5:1-1:1, 10:1-1:1, 25:1-1-1, 50:1-1:1, 100:1-1:1, 500:1-1:1, 1000:1-1:1 or 10,000:1-1:1.

Preferably, a detectably effective amount of the imaging agent of the invention is administered to a subject. In accordance with the invention, “a detectably effective amount” of the imaging agents of the invention is defined as an amount sufficient to yield an acceptable image using equipment, e.g. imaging modalities including MRI and nuclear imaging, which are available for clinical use. A detectably effective amount of the imaging agents of the invention may be administered in more than one injection. The detectably effective amount of the imaging agents of the invention can vary according to factors such as the degree of susceptibility of the individual, the age, sex, and weight of the individual, idiosyncratic responses of the individual, the dosimetry. Detectably effective amounts of the imaging agent of the invention can also vary according to instrument and film-related factors. Optimization of such factors is well within the level of skill in the art. Moreover, two or more imaging agents of the invention can be co-administered in any suitable ratio and in combination with other active ingredients that can be co-administered therewith, such as, anticancer compounds or antibiotics.

The amount of an imaging agent used in accordance with the methods disclosed herein will depend upon the nature of the label domain (e.g. the radionuclide used as the label), the body mass of the patient, the nature and severity, of the condition being treated, the nature of therapeutic treatments which the patient has undergone, and on the idiosyncratic responses of the patient, and the state or status of the MDR phenotype. Ultimately, the attending physician will decide the amount of imaging agent to administer to each individual patient and the duration of the imaging study.

Kits and/or Pharmaceutical Packages

In another embodiment, the present invention provides for kits comprising the novel imaging agents disclosed herein for use in various purposes such as, but not limited to, the detection and/or imaging and/or diagnosis of a cancer having a multidrug resistance phenotype. The kits can be used during, before or after an anticancer treatment and can be used in both in vivo and in vitro applications. In vivo application can include, but are not limited to, detecting and/or imaging and/or diagnosing in a subject undergoing an anticancer treatment a cancer having an multidrug resistance phenotype. The kits can be used in in vitro settings, for example, detecting and/or diagnosing a multidrug phenotype in a cancer cell and/or tissue which has been obtained, for example, through biopsy.

The kits contemplated by the invention can comprise one or more imaging agents of the invention. The kits can also comprise, together or separate from the imaging agents of the invention, additional active ingredients useful in treating a cancer or a condition associated with multidrug resistance, such as, for example, a bacterial infection. Such additional active ingredients can include any known chemotherapeutic agent or any known antibiotic. The kits can comprise any suitable container comprising any compound of the invention as described herein previously or within the ambit of the invention. The kits may also include instructions for using the compounds of the invention in the methods described herein. The kits can also include the pharmaceutical compositions of the invention described herein and can include instructions and any devices which are necessary or advantageous or useful for the administration of the pharmaceutical compositions or inventive compounds, e.g. a syringe or delivery implement. The container is not intended to be limited to any particular form, shape, or size and its construction can be of any suitable material in the art that is not detrimental to the contents contained therein.

All the essential materials and reagents required for administering the compounds of the invention can be assembled together in the herewith kits. When the components of the kit are provided in one or more liquid solutions, the liquid solution preferably is an aqueous solution, with a sterile aqueous solution being particularly preferred.

The components of these kits may be provided in dried or lyophilized forms. When reagents or components are provided in dried form, reconstitution generally is by the addition of a suitable solvent. It is envisioned that the solvent also may be provided in another container means. The kits of the invention may also include an instruction sheet defining administration of the compounds of the invention or for explaining the desired procedures contemplated by the present invention, such as, for example, the diagnosis and/or detection and/or imaging of a multidrug resistant cancer.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of reactions in vitro.

In another embodiment, the kits of the invention may contain the imaging compounds which have been covalently or non-covalently combined with a chelating agent; an auxiliary molecule such as mannitol, gluconate, glucoheptonate, tartrate, and the like; and a reducing agent such as SnCl₂, Na dithionite or tin tartrate. The imaging compound/chelating agent and the auxiliary molecule may be present as separate components of the kit or they may be combined into one kit component. The unlabeled imaging compound/chelating agent, the auxiliary molecule, and the reducing agent may be provided in solution or in lyophilized form, and these components of the kit of the invention may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form.

Determination and optimization of such stabilizers and stabilization methods are well within the level of skill in the art. When the imaging compound/chelating agent of this embodiment are in lyophilized form, the kit may optionally contain a sterile and physiologically acceptable reconstitution medium such as water, saline, buffered saline, and the like. The amounts of imaging compound/chelating agent, auxiliary molecule, and reducing agent in this embodiment are optimized in accordance with the methods for making the images of the multidrug phenotype described herein.

Imaging agents of the invention may be used in accordance with the methods of the invention by one of skill in the art, e.g., by specialists in nuclear medicine, to image cancerous sites having or suspected of having a multidrug resistance phenotype. Any site displaying the multidrug phenotype may be imaged by the imaging methods and imaging agents of the present invention.

The present invention also provide packaged pharmaceutical compositions comprising a pharmaceutical acceptable carrier and a compound or salt of any one of the herein disclosed compounds, including that of Formula 1. In certain embodiments the packaged pharmaceutical composition will comprise the reaction precursors necessary generate the compound or salt according to Formula 1 or subformula thereof. Other packaged pharmaceutical compositions provided by the present invention further comprise indicia comprising at least one of: instructions for using the composition to image cells or tissues bearing a multidrug resistance phenotype or instructions for using the composition to image multidrug resistance in a patient suffering from a cancer, or instructions for using the compositions of the invention to image and/or diagnose a multidrug resistance phenotype during an anticancer treatment followed by the modification and/or improvement of the anticancer treatment to avoid or mitigate the effects of the MDR phenotype, thereby improving the cancer treatment.

### EXAMPLES

The invention will now be further described by way of the following non-limiting examples. Abbreviations used are as follows: ATP binding cassette (ABC); flow cytometry (FACS); magnetic resonance (MR); magnetic resonance imaging (MRI); multi-drug resistance (MDR); multi-drug resistance associated proteins (MRPs); P-glycoprotein (P-gp); positron emission tomography (PET); single photon emission computer tomography (SPECT); and carboxytetramethylrhodamine (TAMRA).

In addition, the following Examples were carried out with the following methods.

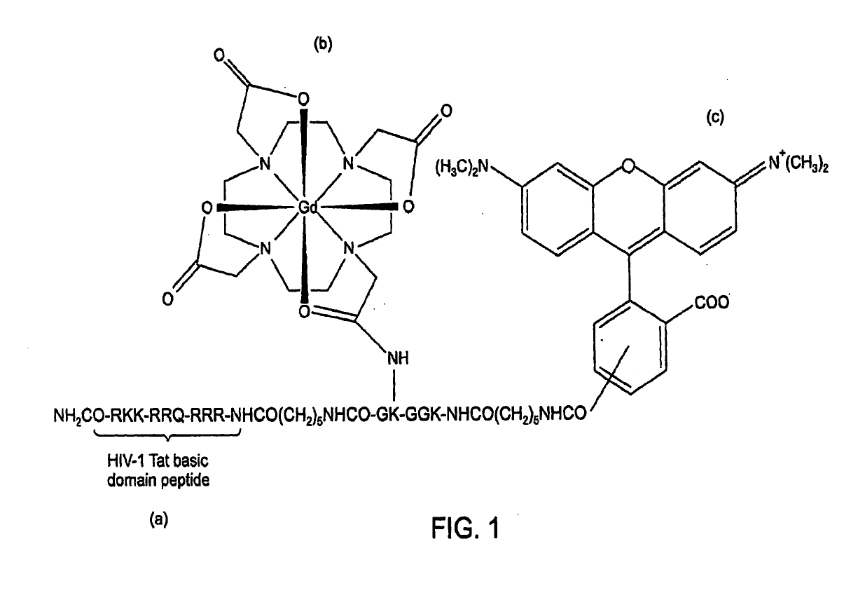

Materials. An HIV-1 Tat basic domain peptide-GdDOTA-carboxytetramethylrhodamine conjugates (Tat-GdDOTA-TAMRA, MW=2,989; as shown in FIG. 1) was custom-synthesized by Global Peptide Services, LLC (Fort Collins, Colo., U.S.A.). The specific T₁ relaxivity (R,) of the agent at 9.4T is 4.9 (sec·mM)⁻¹. Monoclonal anti-P-gp [P-7965], streptavidin-QR conjugates [S-2899], and rhodamine 123 [R-8004] were purchased from Sigma Chemical Company (St. Louis, Mo., U.S.A.). Heptakis-(2,6-di-O-methyl)-β-cyclodextrin (DMCD) and cyclosporin A were purchased from Sigma-Aldrich Co. (St. Louis, Mo., U.S.A.). 4′,6-diamidino-2-phenylindole (DAPI) was obtained from Invitrogen Corp. (Carlsbad, Calif., U.S.A.). All other chemicals were obtained as reagent-grade products.
Cell lines. Human breast adenocarcinoma (MCF-7^{wt}) and its multi-drug-resistant variant (MCF-7^{adr}) were maintained in Eagle's minimum essential medium (EMEM) with 1% penicillin, streptomycin, and 10% fetal bovine serum at 37° C. with 5% CO₂. For MCF-7^{adr} cells, the growth medium was supplemented with 1.5 μM of adriamycin every medium change.
Flow cytometry and fluorescent microscopy. Initially, to check P-gp expression and P-gp function of both cell lines, flow cytometry was implemented using a double staining technique as follows. Briefly, MCF-7^{wt} and MCF-7^{adr} cells were detached from the flask using dissociation buffer, washed with PBS, and incubated with rhodamine 123 for 30 min in the 5% CO₂ incubator. After consecutive washing with PBS, cells were incubated with the biotinylated anti-P-gp antibody for 1 h, followed by 30 min incubation with streptavidin-QR (Quantum Red) conjugates. The biotinylated anti-P-gp antibody was synthesized using EZ-Link®Sulfo-NHS-LC-biotin according to manufacturer's instructions (Pierce Biotechnology, Inc., Rockford, Ill., U.S.A.). After the fixation of cells with 2% paraformaldehyde, flow cytometry was carried out using band-selective emission filters centered around 530 nm for rhodamine 123 and 680 nm for Quantum Red detection.

In addition to the above experiments, collected cell pellets were incubated with 250 μl of Tat-GdDOTA-TAMRA (0.5 mg/ml in EMEM) for 20 min at 37° C. in the 5% CO2 incubator. After consecutive washing with PBS, cells were fixed in 3% paraformaldehyde for 15 min. As a control, cells were incubated with EMEM alone. To inhibit multidrug resistance mechanisms, 100 μM of DMCD was added to cells for 20 min prior to exposure to Tat-GdDOTA-TAMRA. In each step, cells were washed thoroughly. TAMRA fluorescence was detected in the orange-red region using band-selective emission filters centered around 600 nm.

Confocal microscopy. MCF-7^{wt} and MCF-7^{adr} cell lines were separately seeded in each 4-chamber glass slide (Nalge Nunc International Corp., Naperville, Ill., U.S.A.). Cells were incubated with 250 μl of Tat-GdDOTA-TAMRA (10 μg/ml) for 30 min in the 5% CO₂ incubator. Cell nuclei were stained with DAPI (2 pg/ml) for 5 min. Cells were fixed with 3% paraformaldehyde for 15 min, and mounted under a coverslip following removal of plastic chambers. Confocal studies were performed with an inverted Zeiss LSM 410 confocal microscope (Carl Zeiss AG, Jena, Germany) using a 40× water immersion lens.
In vitro MRI study. For the in vitro MRI study, the cell labeling procedure followed the protocol used for flow cytometry and microscopy studies, except that 20 M of cyclosporin A was used, rather than DMCD as a multidrug resistance inhibitor. Cell suspensions were transferred into micro-centrifuge PCR tubes. MRI was carried out on a Bruker horizontal bore 9.4T spectrometer (Bruker Biospin GmbH, Rheinstetten, Germany) equipped with shielded gradients. The Paravision 3.0.2 program (Bruker Biospin GmbH) was used as acquisition software. Images of 8 slices (slice thickness of 1 mm) with an in-plane spatial resolution of 0.3125×0.250 mm (128×64 matrix zero-filled to 128×28, field of view=40×32 mm) were obtained with 8 repetitions. A saturation recovery snapshot-FLASH pulse sequence with an excitation pulse flip angle of 10 degrees, an echo time of 1.245 ms, and ten T₁, saturation recovery delays (50, 100, 250, 500, 1,000, 1,500, 2,000, 3,000, 5,000, 12,000 ms) were used (Bhujwalla, Z. M., Artemov, D., Natarajan, K., Ackerstaff, E. & Solaiyappan, M., “Vasuclar Differences Detected by MRI for Metastatic Versus Nonmetastatic Breast and Prostate Cancer Xenografts”, Neoplasia 3, 143-153 (2002)). Quantitative T₁, maps of the samples were reconstructed using custom-written software in the IDL programming environment Research Systems Inc., Boulder, Colo., U.S.A.) that runs on Linux workstations. Final analysis was performed with the Image) program. (NIH, Bethesda, Md., U.S.A.).

### Example 1

### Design of the Novel Contrast Agent, Tat-GdDOTA-TAMRA

Here, a magnetic resonance (MR) contrast agent for molecular imaging of multidrug resistance (MDR) was designed. This novel magnetic resonance imaging (MRI) contrast agent to detect MDR cancer was studied in model cell systems, and demonstrated differential uptake in wild type and drug resistant human breast cancer cell lines, as shown in the following examples.

It has been reported that [^{99m}Tc]SESTAMIBI is retained in the wild type KB tumor, but not in the drug-resistant phenotype despite similar perfusion (Piwnica-Worms, D., et al., “Functional Imaging of Multidrug-Resistant P-Glycoprotein with an Organotechnetium Complex”, Cancer Res 53, 977-984 (1993)). This effect was attributed to P-gp-mediated transport of [^{99m}Tc]SESTAMIBI from the cell in MDR tumors in vivo. While the SPECT-MIBI protocol is currently approved for clinical use, there are several potential problems associated with nuclear imaging methods. Further, [^{99m}Tc]MIBI is a generic agent, and its uptake and efflux kinetics may not be optimal for MDR detection.

Putative MRI-detectable, MDR-specific contrast agents theoretically should (i) efficiently internalize into cancer cells, (ii) contain a contrast-generating moiety that renders the agent detectable by MRI, (iii) act as a substrate for MDR receptors and be specifically exported from MDR cells via these receptors, (iv) have no or low toxicity, and (v) be sufficiently stable in vivo. An innovative idea in the design of low molecular weight intracellular gadolinium paramagnetic contrast agents was to link the Gd complex to an amphiphilic membrane-crossing transport peptide, such as the HIV-1 virus Tat basic domain (Prantner, A. M., Sharma, V., Garbow, J. R. & Piwnica-Worms, D., “Synthesis and Characterization of a Gd-DOT-a-d Permeation Peptide for Magetic Resonance Relaxation Enhancement of Intracellular Targets”, Mol. Imaging. 2, 333-341 (2003); Bhorade, R., Weissleder, R., Nakakoshi, T., Moore, A. & Tung, C. H., “Macrocyclic Chelators with Paramagnetic cations are Internalized into Mammalian Cells Via a HIV-tat Derived Membrane Translocation Peptide”, Bioconjug Chem 11, 301-305 (2000)). Using all D-amino acids and/or using poly-arginine as a transport peptide may improve the efficiency of contrast agent transport across the cellular membrane and improve stability in vivo (Prantner, A. M., Sharma, V., Garbow, J. R. & Piwnica-Worms, D., “Synthesis and Characterization of a Gd-DOT A-D-Permeation Peptide for Magnetic Resonance Relaxation Enhancement of Intracellular Targets”, Mol Imaging 2, 333-341 (2003); Allen, M. J. & Meade, T. J., “Synthesis and Visualization of a Membrane-Permeable MRI Contrast Agent”, J. Biol Inorg Chem 8, 746-750 (2003); Wender, P. A. et al., “The Design, Synthesis, and Evaluation of Molecules That Enable or Enhance Cellular Uptake Peptoid Molecular Transporters”, Proc Natl Acad Sci USA 97, 13003-13008 (2000); Gammon, S. T., Villalobos, V. M., Prior, J. L., Sharma, V. & Piwnica-Worms, D., “Quantitative Analysis of Permeation Peptide Complexes Labeled With Technetium-99m: Chiral and Sequence-Specific Effects on Net Cell Uptake”, Bioconjug Chem 14, 368-376 (2003)). Gd³⁺ ion was complexed by a polycyclic chelate, DOTA, that provides the highest stability for the complex (Corot, C. et al., “Structure-Activity Relationship of Macrocyclic and Linear Gadolinium Chelates: Investigation of Transmetallation Effect on the Zinc-Dependent Metallopeptidase Angiotensin-Converting Enzyme”, J Magn Reson Imaging 8, 695-702 (1998)). To enable MDR-specific efflux of the agent from drug-resistant cells, an additional structure that may serve as a substrate for the P-gp transporter is required.

An HIV-1 Tat basic domain peptide-GdDOTA-carboxytetramethylrhodamine conjugates (Tat-GdDOTA-TAMRA, MW=2,989; as shown in FIG. 1) was used in these studies. The novel contrast agent, Tat-GdDOTA-TAMRA, was designed using solid-state oligopeptide synthesis technology, and conjugated to a peptide backbone containing the HIV-1 Tat basic domain to the GdDOTA complex through a flexible linker, aminohexanoic acid, and to a TAMRA red fluorescent probe. As shown in FIG. 1, the structure of the contrast agent has several functional domains, each playing a particular each playing a particular role for multidrug resistant (MDR)-specific magnetic resonance (MR) imaging. FIG. 1(A) shows the chemical structure and (B) concept of the Tat-GdDOTA-TAMRA contrast agent. The HIV-1 Tat basic domain peptide, GdDOTA, and carboxytetramethylrhodamine (TAMRA) were used as an amphiphilic membrane translocation oligopeptide, a Gd chelate complex, and a substrate for the specific drug-resistant transporter, respectively.

### Example 2

### Testing of Contrast Agent to Detect Multidrug Resistant Cells

Flow Cytometry and Fluorescent Microscopy

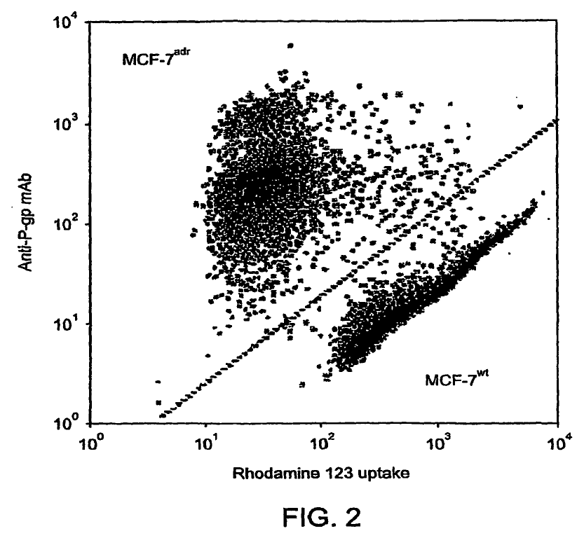

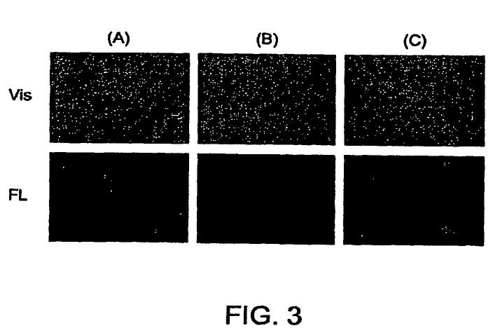

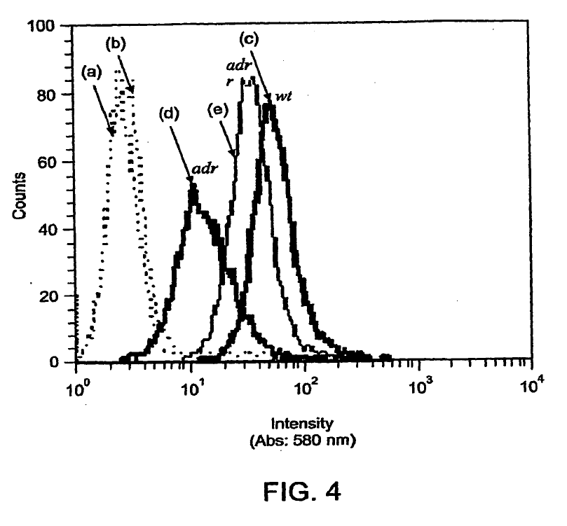

Here, the P-glycoprotein (P-gp)expression and the function of both cell lines are shown in FIG. 2. P-gp/MDR1 were overexpressed in the drug-resistant MCF-7^{adr} cells, and a reduced accumulation of rhodamine 123 was observed in the drug-resistant cells, due to the active efflux of the maker by ABC transporters, especially P-gp (Twentyman, P. R., Rhodes, T. & Rayner, S. A., “A Comparison of Rhodamine 123 Accumulation and Efflux in Cells with P-Glycoprotein-Mediated and MRP-Associated Multidrug Resistance Phenotypes”, Eur J Cancer 30A, 1360-1369 (1994)). MCF-7^{wt} cells had a low P-gp expression, and demonstrated an efficient accumulation of rhodamine 123. Results of fluorescent microscopy of MCF-7^{wt} and MCF-7^{adr} cells treated with a contrast agent, Tat-GdDOTA-TAMRA, are shown in FIG. 3. Efficient uptake of the agent by MCF-7^{wt} cells resulted in bright fluorescence images of the cells, as shown in FIG. 3A. Lower or no fluorescence was detected in the MCF-7^{adr} cells, as seen in FIG. 3B, thus showing that the cells efficiently exported the agent through MDR transporters. Pre-treatment of MCF-7^{adr} cells with the MDR inhibitor, heptakis-(2,6-di-O-methyl)-β-cyclodextrin (DMCD), led to the high retention of the conjugates, as shown in FIG. 3C. Tat-GdDOTA-TAMRA uptake in the MCF-7^{wt} and MCF-7^{adr} cells obtained by FACS analysis is shown in histograms of FIG. 4. FIG. 4 shows that the agent delivered into MCF-7 dr cells was significantly lower in comparison to MCF-7^{wt}′ cells due to the MDR transporter. Indeed, the uptake of the agent was recovered almost to the level typical for wild type cells by pre-treatment of MCF-7^{adr} with the MDR inhibitor, DMCD. Overall, Tat-GdDOTA-TAMRA was significantly better retained in MCF-7′ cells than in MCF-7^{adr} cells,

Confocal Microscopy

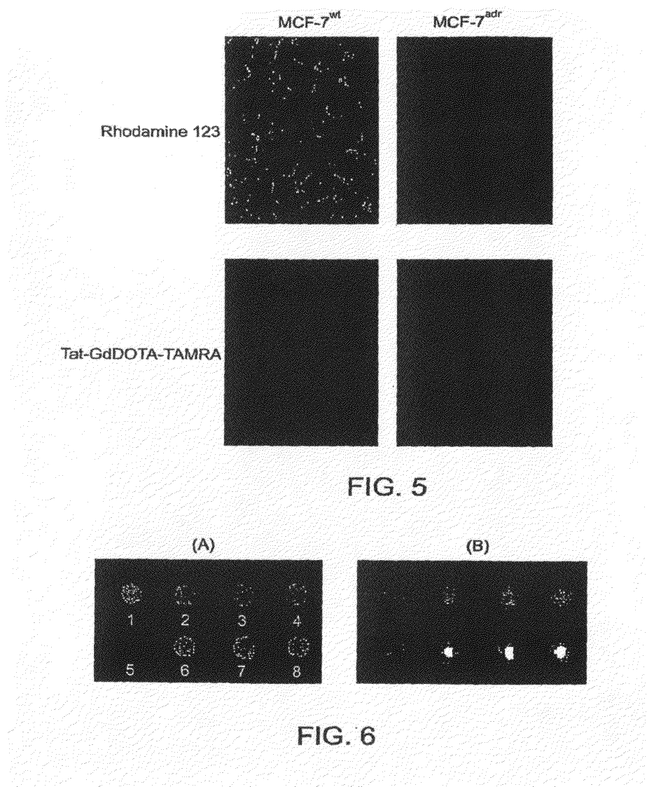

Confocal microscopy images of MCF-7^{wt} and MCF-7^{adr} cells treated with Tat-GdDOTA-TAMRA and 4′,6-diamidino-2-phenylindole (DAPI) are provided in FIG. 5. The agents were co-localized with nuclei in wild type cells, whereas significantly less compound and less co-localization with nuclei was detected in MDR cells.

In Vitro MRI

T₁ relaxation maps and intensity maps of MCF-7^{wt} and MCF-7^{adr} cell samples are shown in FIG. 6. Averaged T₁ values for different samples are presented in Table 1, shown below. The agents significantly decreased T₁ relaxation time in wild type cells, whereas almost no decrease in T₁, relaxation was observed in MDR cells treated with the conjugates alone, which indicated that Tat-GdDOTA-TAMRA was retained in MCF-7^{adr} cells but not in MCF-7^{adr} cells. A significant shortening of T₁ relaxation time was observed following pre-treatment of MCF-7^{adr} cells with the MDR inhibitor, cyclosporin A. No relaxation analysis was performed for the water sample doped with the GdDTPA relaxation agent (#5 on Table 1), as both T₁ and T₂ relaxation times of this sample were on the order of 25 ms.

TABLE 1
T1 values of water, GdDTPA, and cell samples

| | #* | | | |
|---|---|---|---|---|
| | | 2 | 3 | 4 |
| | | MCF-7^{wt} | | |
| | | | | Tat-GdDOTA- |
| | 1 | | Tat-GdDOTA- | TAMRA + |
| Sample | Water | Control | TAMRA | Cyclosporin A |
| T₁ (s) | 2.71 ± 0.07 | 2.33 ± 0.08 | 1.79 ± 0.02 | 1.79 ± 0.04 |

| | #* | | | |
|---|---|---|---|---|
| | | 6 | 7 | 8 |
| | | MCF-7^{adr} | | |
| | | | | Tat-GdDOTA- |
| | 5 | | Tat-GdDOTA- | TAMRA + |
| Sample | GdDTPA | Control | TAMRA | Cyclosporin A |
| T₁ (s) | N/A | 2.34 ± 0.05 | 2.05 ± 0.06 | 1.91 ± 0.02 |

*Each sample number (#) corresponds to the one in FIG. 6.

An important result that can be seen from Table 1 is that the novel agent, Tat-GdDOTA-TAMRA, can efficiently discriminate between MCF-7^{wt} and MCF-7^{adr} cells using T₁ MRI. T₁ relaxation time was significantly reduced in MCF-7′ cells treated with 0.5 mg/ml of Tat-GdDOTA-TAMRA. T₁ reduction in MCF-7^{adr} cells (ΔT₁=0.3±0.11 s, see Table 1 columns 6 and 7) was significantly lower than that of MCF-7^{wt} (ΔT₁=0.54±0.11 s, see Table, 1 columns 2 and 3). Further, increased uptake of the conjugates in the MDR inhibitor-treated MCF-7^{adr} cells suggests involvement of MDR transporters in the efflux of the Tat-GdDOTA-TAMRA agent from MCF-7^{adr} cells that overexpress P-gp protein (see, for example Table 1 and FIGS. 3, 4, and 6).

Taken together, the data presented herein shows that the Tat/Gd/substrate-based molecular probe was accumulated efficiently in the wild type tumor cells while the drug resistant phenotype efficiently pumped out this agent from the cells in vitro. This was confirmed by flow cytometry, microscopy, and MRI T₁ measurements.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### REFERENCES

1. Neyfakh, A. A. (2002) Mol Microbiol 44, 1123-1130.
2. Bodo, A., Bakos, E., Szeri, F., Varadi, A. & Sarkadi, B. (2003) Toxicol Lett 140-141, 133-143.
3. Chiu, M. L., Kronauge, J. F. & Piwnica-Worms, D. (1990) J Nucl Med 31, 1646-53.
4. Piwnica-Worms, D., Chiu, M. L., Budding, M., Kronauge, J. F., Kramer, R. A. & Croop, J. M. (1993) Cancer Res 53, 977-84.
5. Twentyman, P. R., Rhodes, T. & Rayner, S. (1994) Eur J Cancer 30A, 1360-1369.
6. Bhujwalla, Z. M., Artemov, D., Natarajan, K., Ackerstaff, E. & Solaiyappan, M. (2001) Neoplasia 3,143-153.
7. Piwnica-Worms, D., Chiu, M. L., Budding, M., Kronauge, J. F., Kramer, R. A. & Croop, J. M. (1993) Cancer Res 53, 977-984.
8. Prantner, A. M., Sharma, V., Garbow, J. R & Piwnica-Worms, D. (2003) Mol Imaging 2, 333-41.
9. Bhorade, R., Weissleder, R., Nakakoshi, T., Moore, A. & Tung, C. H. (2000) Bioconjug Chem 11, 301-5.
10. Allen, M. J. & Meade, T. J. (2003) J Biol Inorg Chem 8, 746-50.
11. Wender, P. A., Mitchell, D. J., Pattabiraman, K., Pelkey, E. T., Steinman, L. & Rothbard, J. B. (2000) Proc Natl Acad Sci USA 97, 13003-8.
12. Gammon, S. T., Villalobos, V. M., Prior, J. L., Sharma, V. & Piwnica-Worms, D. (2003) Bioconjug Chem 14, 368-76.
13. Corot, C., Idee, J. M., Hentsch, A. M., Santus, R., Mallet, C., Goulas, V., Bonnemain, B. & Meyer, D. (1998) J Magn Reson Imaging 8, 695-702.
14. Torchilin, V. P., Levchenko, T. S., Rammohan, R., Volodina, N., Papahadjopoulos-Sternberg, B. & D'Souza, G. G. (2003) Proc Natl Acad Sci USA 100, 1972-7.
15. Zhao, M., Kircher, M. F., Josephson, L. & Weissleder, R. (2002) Bioconjug Chem 13, 8404.
16. Merdan, T., Kope\check{c}ek, J. & Kissel, T. (2002) Adv Drug Deliv Rev 54, 715-758.
17. Prantner, A. M., Sharma, V., Garbow, J. R. & Piwnica-Worms, D. (2003). Mol Imaging 2, 333-341.

1. An agent for imaging a multidrug resistance phenotype in a cancer cell, said agent comprising a transduction domain capable of translocating the agent into the cancer cell, a label domain capable of being detected, and a substrate domain capable of functioning as a substrate for a multidrug resistance transporter.

2. The agent according to claim 1, wherein the transduction domain is an amphiphilic membrane translocation peptide.

3. The agent according to claim 2, wherein the amphiphilic membrane translocation peptide is HIV-1 Tat basic domain peptide.

4. The agent according to claim 1, wherein the transduction domain is selected from the group consisting of HIV-1 Tat basic domain peptide, a Drosophila Antennapedia protein transduction domain, a polyarginine, and a polylysine.

5. The agent according to claim 1, wherein the label domain is a magnetic resonance contrast agent suitable for magnetic resonance imaging.

6. The agent according to claim 5, wherein the magnetic resonance contrast agent comprises a paramagnetic metal chelate.

7. The agent according to claim 6, wherein the paramagnetic metal chelate is a gadolinium-based chelate.

8. The agent according to claim 5, wherein the paramagnetic metal chelate comprises a paramagnetic metal selected from the group consisting of Gd(III), Fe(III), Mn(II and III), Cr(III), Cu(II), Dy(III), Tb(III), Ho(III), Er(III), and Eu(III).

9. The agent according to claim 5, wherein the paramagnetic metal chelate comprises a chelator selected from the group consisting of HYNIC, DRPA, EDTA, DOTA, TETA, DTPA and BAT.

10. The agent according to claim 1, wherein the label domain is a radionuclide suitable for nuclear imaging.

11-15. (canceled)

16. A compound or pharmaceutically acceptable salt thereof for detecting a multidrug resistance phenotype according to Formula 1:
[Y¹—Y³]—Y²
wherein
Y¹ is a transduction peptide,
Y² is a contrast agent capable of being detected by an imaging modality, and
Y³ is a multidrug resistance transporter substrate.

17. A pharmaceutical composition comprising a pharmaceutically acceptable carrier and the compound according to claim 16.

18. A package comprising the pharmaceutical composition of claim 17 in a container and further comprising indicia comprising at least one of (a) instructions for using the composition to image cells or tissues having a multidrug resistance phenotype, or (b) instructions for using the composition to predict effectiveness of treatment with a chemotherapeutic agent, or (c) instructions for using the composition to selectively image cells having a multidrug resistance phenotype.

19. A method for detecting a cancer cell having a multidrug resistance phenotype comprising providing an imaging agent, contacting the cancer cell with the imaging agent, and making an image using a medical imaging modality, wherein the imaging agent comprises a transduction domain capable of translocating the agent into the cancer cell, a label domain capable of being detected by the medical imaging modality, and a substrate domain capable of being transported by a multidrug resistance transporter.

20. The method of claim 19, wherein the method for detecting is carried out in vivo in a subject undergoing a chemotherapeutic treatment.

21-44. (canceled)

45. A method for preventing or reducing multidrug resistant cancer in a subject undergoing treatment with a chemotherapeutic agent comprising administering to the subject an imaging agent in an amount sufficient to detect in the subject a multidrug resistance phenotype if present, making an image using a medical imaging modality, reading the image to detect a multidrug resistance phenotype if present, and optimizing the chemotherapeutic treatment if a multidrug resistance phenotype is detected thereby preventing or reducing the multidrug resistant cancer in the subject, wherein the imaging agent comprises a transduction domain capable of translocating the agent into the cancer cell, a label domain capable of being detected by the medical imaging modality, and a substrate domain capable of being transported by a multidrug resistance transporter.

46. The method of claim 45, wherein the multidrug resistance phenotype is associated with an ATP binding cassette transporter (ABC), a multidrug resistance associated protein (MRP), or an ABC half-transporter (ABCG2).

47-66. (canceled)

67. (canceled)

68-85. (canceled)

86. An imaging method comprising the steps of:
providing an imaging compound according to Formula 1:
[Y¹—Y³]—Y²
wherein,
Y¹ is a transduction domain;
Y² is a contrast agent capable of being detected by an imaging modality; and
Y³ is a multidrug resistance transporter substrate;
contacting cells or tissues with the compound; and
making an image with an imaging modality.

87-101. (canceled)
